(19) United States
(12) Patent Application Publication
(10) Pub. No.: US 2025/0281172 A1
(43) Pub. Date: Sep. 11, 2025

(54) MEDICAL SYSTEM AND SUTURE DEVICE

(71) Applicant: OLYMPUS CORPORATION, Tokyo (JP)

(72) Inventors: Michael BLOM, Cambridge (GB); Alex NORMAN, Cambridge (GB); Mark RIDLEY, Cambridge (GB); Andrew FORBES, Cambridge (GB); Merissa LIM, Cambridge (GB); Antonia SMITH, Cambridge (GB); Gary STACEY, Cambridge (GB); Shintaro INOUE, Westborough, MA (US); Ryohei OGAWA, Hachioji-shi (JP)

(73) Assignee: OLYMPUS CORPORATION, Tokyo (JP)

(21) Appl. No.: 19/069,448

(22) Filed: Mar. 4, 2025

Related U.S. Application Data
(60) Provisional application No. 63/562,855, filed on Mar. 8, 2024.
(60) Provisional application No. 63/635,021, filed on Apr. 17, 2024.
(60) Provisional application No. 63/648,937, filed on May 17, 2024.
(60) Provisional application No. 63/664,879, filed on Jun. 27, 2024.
(60) Provisional application No. 63/669,306, filed on Jul. 10, 2024.
(60) Provisional application No. 63/688,972, filed on Aug. 30, 2024.
(60) Provisional application No. 63/691,009, filed on Sep. 5, 2024.
(60) Provisional application No. 63/695,602, filed on Sep. 17, 2024.
(60) Provisional application No. 63/737,339, filed on Dec. 20, 2024.

(51) Int. Cl. A61B 17/04; A61B 1/005; A61B 1/012; A61B 1/05; A61B 17/00; A61B 17/06; A61B 17/062

(52) U.S. Cl. CPC A61B 17/0469; A61B 1/005; A61B 1/0125; A61B 1/05; A61B 17/0401; A61B 17/0467; A61B 17/06004; A61B 17/06066; A61B 17/0625; A61B 2017/00862; A61B 2017/0409; A61B 2017/0414; A61B 2017/0416; A61B 2017/0454; A61B 2017/047; A61B 2017/06019; A61B 2017/0608

(57) ABSTRACT

A medical system includes a suture device. The suture device includes: an arm; a first jaw and a second jaw that are attached to a distal end of the arm; and a needle configured to attach a thread. At least one of the first jaw and the second jaw configured to be opened and closed to deliver the needle therebetween.

### CROSS-REFERENCE TO RELATED APPLICATIONS

Priority is claimed on U.S. Provisional Patent Applications (1) No. 63/562,855 filed on Mar. 8, 2024, (2) No. 63/635,021 filed on Apr. 17, 2024, (3) No. 63/648,937 filed on May 17, 2024, (4) No. 63/664,879 filed on Jun. 27, 2024, (5) No. 63/669,306 filed on Jul. 10, 2024, (6) No. 63/688,972 filed on Aug. 30, 2024, (7) No. 63/691,009 filed on Sep. 5, 2024, (8) No. 63/695,602 filed on Sep. 17, 2024, and (9) No.

63/737,339 filed on Dec. 20, 2024, the contents of which are incorporated herein by reference.

### FIELD

The present invention relates to a medical system and a suture device.

### BACKGROUND

In the related art, a medical manipulator system for observation or treatment in a hollow organ such as the alimentary canal has been used. The medical manipulator system can electrically drive an insertion section or the like which is inserted into a hollow organ. A user can control the operation of the insertion section or the like using an operation device which is provided outside of an internal part.

In PCT International Publication No. WO 2021/145411 (hereinafter referred to as Patent Document 1), a medical system including an endoscope which is electrically driven is described. In the medical system described in Patent Document 1, since the endoscope is electrically driven, it is possible to reduce fatigue of an operator.

The medical manipulator system according to the related art described in Patent Document 1 is not normally easily used and is not a system that can efficiently perform treatment using a medical manipulator (such as an endoscope).

### SUMMARY

The present invention provides a medical system and a suture device.

A medical manipulator according to a first aspect of the present invention includes a suture device. The suture device includes: an arm; a first jaw and a second jaw that are attached to a distal end of the arm; and a needle configured to attach a thread. At least one of the first jaw and the second jaw configured to be opened and closed to deliver the needle therebetween.

With the medical system and the suture device according to the present invention, it is possible to more efficiently perform observation or treatment.

### BRIEF DESCRIPTION OF THE DRAWINGS

FIG. 1 is a diagram illustrating the entire configuration of an electric endoscope system according to a first embodiment.

FIG. 2 is a diagram illustrating an insertion manipulator of an electric endoscope system which is inserted into a large intestine.

FIG. 3 is a diagram illustrating a distal end portion of an insertion section of the insertion manipulator.

FIG. 4 is a front view of the distal end portion when seen from the distal end side.

FIG. 5 is a sectional view of the distal end portion.

FIG. 6 is a diagram illustrating a bendable portion of the insertion manipulator.

FIG. 7 is a diagram illustrating joint rings of the bendable portion.

FIG. 8 is a diagram illustrating a first channel tube of the insertion manipulator.

FIG. 9 is a sectional view of a proximal end channel tube of the first channel tube.

FIG. 10 is a functional block diagram of a drive device.

FIG. 11 is a functional block diagram of a video control device.

FIG. 12 is a diagram illustrating a modified example of the bendable portion.

FIG. 13 is a diagram illustrating a first channel tube which is inserted into the modified example of the bendable portion.

FIG. 14 is a diagram illustrating a modified example of the insertion section.

FIG. 15 is a diagram illustrating a spiral tube.

FIG. 16 is a diagram illustrating a modified example of the distal end portion.

FIG. 17 is a diagram illustrating a hardness changing device according to a second embodiment.

FIG. 18 is a diagram illustrating the entire configuration of the hardness changing device.

FIG. 19 is a diagram illustrating a hardness changing unit.

FIG. 20 is a diagram illustrating the hardness changing unit.

FIG. 21 is a diagram illustrating an operation of the hardness changing unit.

FIG. 22 is a diagram illustrating an operation of the hardness changing unit.

FIG. 23 is a diagram illustrating an operation of the hardness changing unit.

FIG. 24 is a diagram illustrating an operation of the hardness changing unit.

FIG. 25 is a diagram illustrating an operation of the hardness changing unit.

FIG. 26 is a diagram illustrating a modified example of the hardness changing unit.

FIG. 27 is a diagram illustrating the modified example of the hardness changing unit.

FIG. 28 is a diagram illustrating another modified example of the hardness changing unit.

FIG. 29 is a diagram illustrating the modified example of the hardness changing unit.

FIG. 30 is a diagram illustrating another modified example of the hardness changing unit.

FIG. 31 is a diagram illustrating the modified example of the hardness changing unit.

FIG. 32 is a diagram illustrating another modified example of the hardness changing unit.

FIG. 33 is a diagram illustrating a high-frequency knife in a manipulator tool according to a third embodiment.

FIG. 34 is a diagram illustrating a high-frequency knife for marking.

FIG. 35 is a diagram illustrating a local injection needle.

FIG. 36 is a diagram illustrating the local injection needle that locally injects a liquid.

FIG. 37 is a diagram illustrating the high-frequency knife for incision.

FIG. 38 is a diagram illustrating the high-frequency knife for incision.

FIG. 39 is a diagram illustrating a basket.

FIG. 40 is a diagram illustrating the basket for recovering a target part.

FIG. 41 is a diagram illustrating a manipulator tool according to a fourth embodiment.

FIG. 42 is a sectional view of the manipulator tool.

FIG. 43 is a diagram illustrating a modified example of the manipulator tool.

FIG. 44 is a diagram illustrating an artificial muscle disposed at another position.

FIG. 45 is a diagram illustrating an artificial muscle disposed at another position.

FIG. 46 is a diagram illustrating an artificial muscle disposed at another position.

FIG. 47 is a diagram illustrating an artificial muscle disposed at another position.

FIG. 48 is a diagram illustrating an artificial muscle disposed at another position.

FIG. 49 is a diagram illustrating another modified example of the manipulator tool.

FIG. 50 is a diagram illustrating a suture device according to a fifth embodiment.

FIG. 51 is a diagram illustrating a captured image captured by a scope of the insertion manipulator.

FIG. 52 is a diagram illustrating a needle that is delivered from a first jaw to a second jaw in the suture device.

FIG. 53 is a diagram illustrating the needle that is delivered from the first jaw to the second jaw in the suture device.

FIG. 54 is a diagram illustrating the needle that is delivered from the first jaw to the second jaw in the suture device.

FIG. 55 is a diagram illustrating the needle that is delivered from the first jaw to the second jaw in the suture device.

FIG. 56 is a diagram illustrating a sutured defective part.

FIG. 57 is a diagram illustrating an operation of an anchor applier.

FIG. 58 is a diagram illustrating an operation of the anchor applier.

FIG. 59 is a diagram illustrating an operation of the anchor applier.

FIG. 60 is a diagram illustrating an operation of the anchor applier.

### DETAILED DESCRIPTION

### First Embodiment

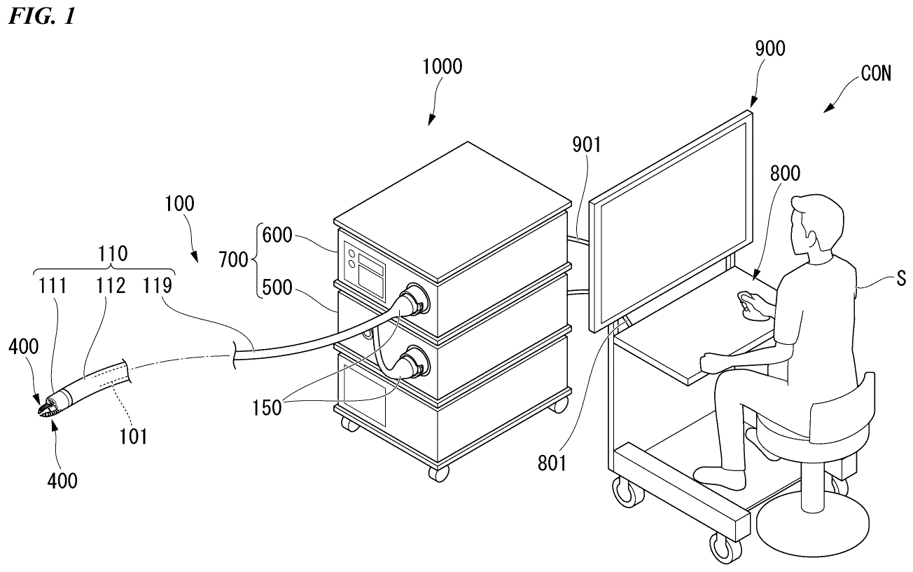

An electric endoscope system 1000 according to a first embodiment of the present invention will be described below with reference to FIGS. 1 to 13. FIG. 1 is a diagram illustrating the entire configuration of the electric endoscope system 1000 according to the present embodiment. The electric endoscope system 1000 is an example of a medical manipulator system. A medical manipulator includes an insertion manipulator 100 which is inserted into a human body and an endoscope, a catheter, a treatment tool, and an endoluminal device which are electrically driven.

### Electric Endoscope System 1000

The electric endoscope system 1000 is a medical system that observes and treats an internal part of a patient. The electric endoscope system 1000 includes an insertion manipulator 100, a treatment manipulator 400, a drive device 500, a video control device 600, an operation device 800, and a display device 900.

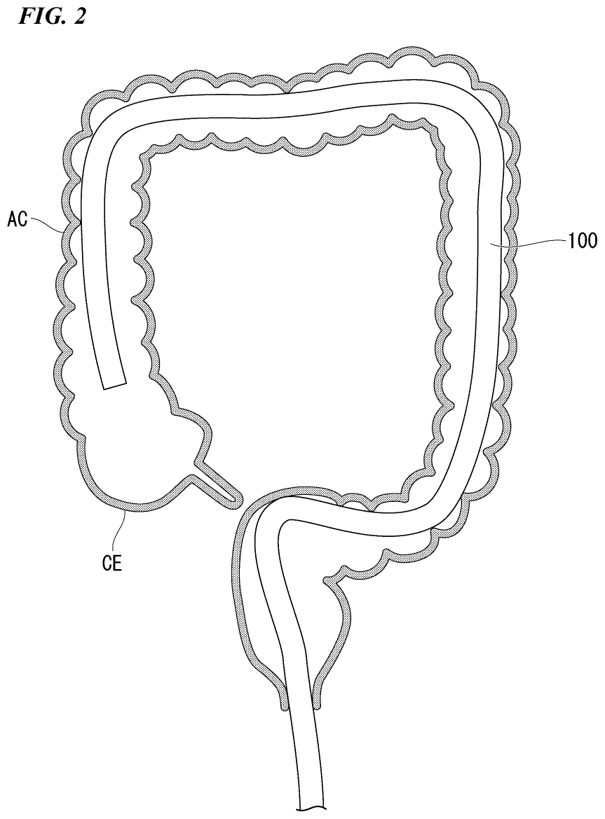

FIG. 2 is a diagram illustrating an insertion manipulator 100 which is inserted into a large intestine.

The insertion manipulator 100 is a device that is inserted into a lumen of a patient to observe and treat a lesion. The insertion manipulator 100 has excellent insertability and can be inserted to, for example, an ascending colon AC or a cecum CE of a large intestine as illustrated in FIG. 2. The insertion manipulator 100 is detachably attached to the drive device 500 and the video control device 600. An internal path 101 is formed in the insertion manipulator 100. In the following description, a side of the insertion manipulator 100 which is inserted into a lumen of a patient is referred to as a “distal end side (distal side) A1,” and a side which is attached to the drive device 500 is referred to as a “proximal side (proximal side) A2.”

The treatment manipulator 400 is, for example, a device that is inserted into a first channel tube 171 of the insertion manipulator 100, protrudes from a first opening 111a, and is inserted into a lumen of a patient to treat a lesion. An end effector (a treatment portion) that treats a lesion is disposed at a distal end of the treatment manipulator 400.

The drive device 500 is detachably connected to the insertion manipulator 100 and the operation device 800. The drive device 500 drives a motor built thereinto on the basis of an operation input to the operation device 800 to electrically drive the insertion manipulator 100. The drive device 500 drives a pump or the like built thereinto on the basis of an operation input to the operation device 800 and causes the insertion manipulator 100 to perform air supply, water supply, and suction.

The video control device 600 is detachably connected to the insertion manipulator 100 and acquires a captured image from the insertion manipulator 100. The video control device 600 causes the display device 900 to display the captured image acquired from the insertion manipulator 100 or a GUI image or a CG image for providing information to an operator.

The drive device 500 and the video control device 600 constitute a control device 700 that controls the electric endoscope system 1000. The control device 700 may further include peripherals such as a video printer. The drive device 500 and the video control device 600 may be a unified device.

The operation device 800 is detachably connected to the drive device 500 via an operation cable 801. The operation device 800 may communicate with the drive device 500 in a wireless communication manner instead of in a wired communication manner. An operator S can electrically drive the insertion manipulator 100 by operating the operation device 800.

The display device 900 is a device that can display an image such as an LCD. The display device 900 is connected to the video control device 600 via a display cable 901.

The operation device 800 and the display device 900 are mounted in a trolley. The trolley on which the operation device 800 and the display device 900 are mounted is also referred to as a “console CON.”

The constituent devices of the electric endoscope system 1000 will be described below.

### Insertion Manipulator 100

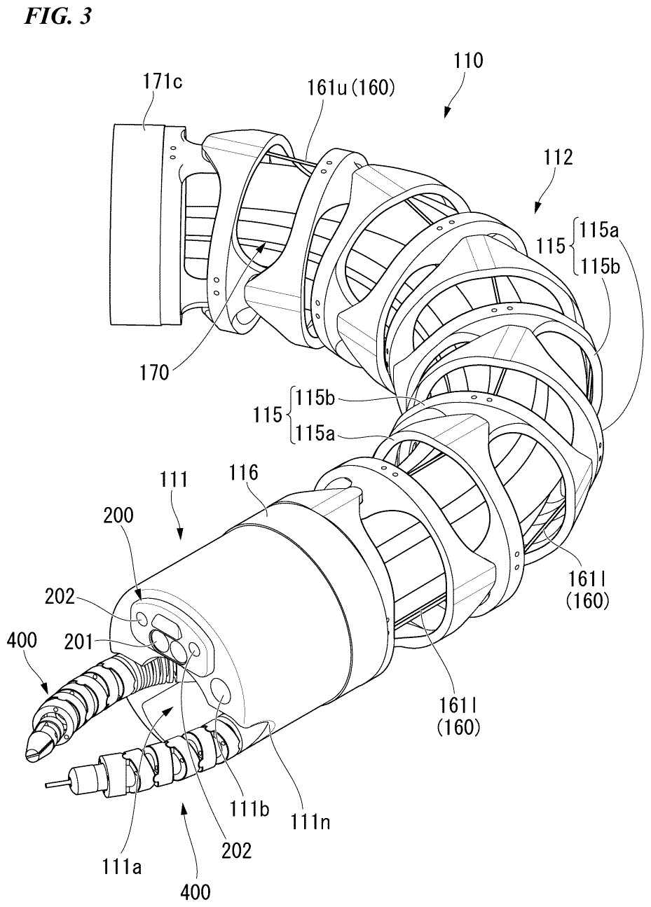

FIG. 3 is a diagram illustrating a distal end portion of an insertion section 110.

The insertion manipulator 100 includes an insertion section 110, an attachment portion 150, a bending wire 160, a built-in element 170, and a scope 200 as illustrated in FIGS. 1 and 3.

In the insertion manipulator 100, an internal path (a lumen) 101 extending in a longitudinal direction (a longitudinal axis direction, an axial direction) A of the insertion manipulator 100 is formed from a distal end of the insertion section 110 to a proximal end of the attachment portion 150. The bending wire 160 and the built-in element 170 are inserted into the internal path 101.

The insertion section 110 is a thin longitudinal member that can be inserted into a lumen. The insertion section 110 includes a distal end portion 111, a bending portion 112, and a flexible portion 119. The distal end portion 111, the bending portion 112, and the flexible portion 119 are sequentially connected from the distal end side A1 to the proximal side A2. The insertion section 110 includes an outer sheath 118 which is disposed outermost.

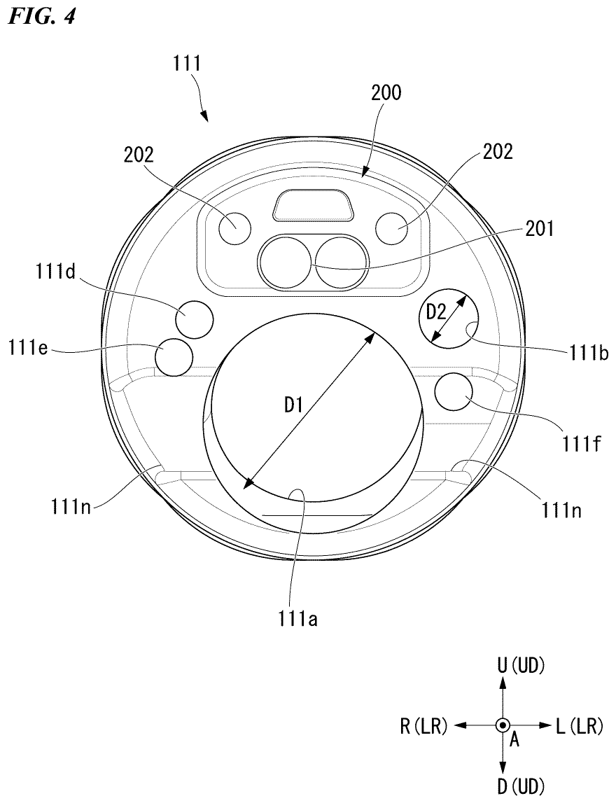

FIG. 4 is a front view of the distal end portion 111 when seen from the distal end side A1.

The distal end portion 111 is formed in a cylindrical shape. The distal end portion 111 includes a first opening 111a, a second opening 111b, a water supply nozzle 111d, an air supply nozzle 111e, and a suction nozzle 111f. The first opening 111a, the second opening 111b, the water supply nozzle 111d, the air supply nozzle 111e, and the suction nozzle 111f are formed on a distal end surface of the distal end portion 111.

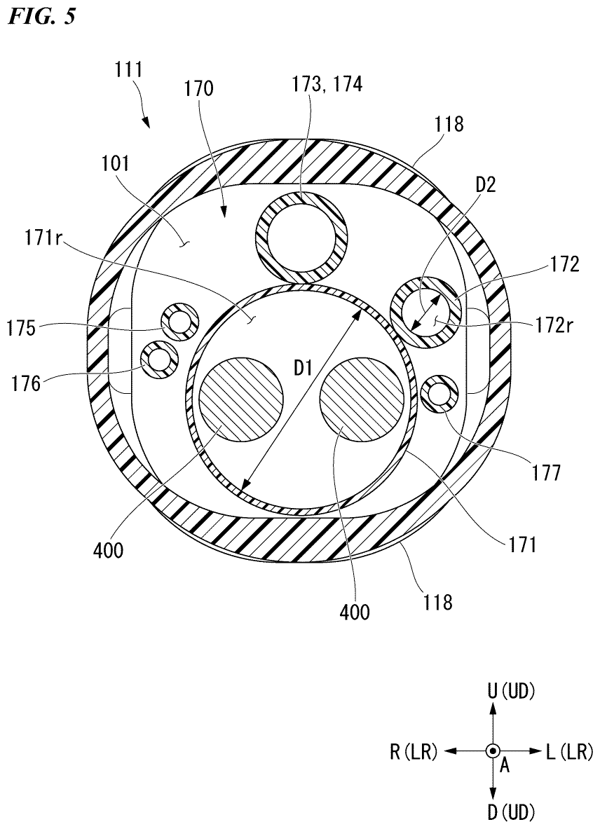

FIG. 5 is a sectional view of the distal end portion 111.

The built-in element 170 is inserted into the internal path 101. The built-in element 170 includes a first channel tube 171, a second channel tube 172, an imaging cable 173, a light guide 174, a water supply tube 175, an air supply tube 176, and a suction tube 177. In FIG. 5, two treatment manipulators 400 that are inserted into the first channel tube 171 are also illustrated.

The first opening 111a is an opening that communicates with the first channel tube 171. The first opening 111a is a circular opening in a front view when seen from the distal end side A1. The distal end of the treatment manipulators 400 inserted into the first channel tube 171 protrudes from and retract to the first opening 111a.

In the first opening 111a, a cutout portion 111n is formed at both ends in a direction (an LR direction which will be described later) perpendicular to a longitudinal direction A. As illustrated in FIG. 3, the treatment manipulators 400 protruding from the first opening 111a to the distal end side A1 can be inserted into the cutout portions 111n. The cutout portions 111n may not penetrate in the direction perpendicular to the longitudinal direction A as long as they are formed on the inner circumferential surface of the first opening 111a.

The second opening 111b is an opening that communicates with the second channel tube 172. The second opening 111b is a circular opening in a front view when seen from the distal end side A1. A distal end of the treatment manipulator 400 inserted into the second channel tube 172 protrudes from and retracts to the second opening 111b. Devices inserted into the second channel tube 172 are not limited to the treatment manipulator 400 and include medical devices such as manual instruments.

An inner diameter D1 of a part other than the cutout portions 111n in the first opening 111a is larger than an inner diameter D2 of the second opening 111b. Specifically, the diameter D1 of the part other than the cutout portions 111n in the first opening 111a is three to five times the inner diameter D2 of the second opening 111b.

The water supply nozzle 111d is an opening that communicates with the water supply tube 175. A liquid in a tank installed in the vicinity of the control device 700 is sent out from the water supply nozzle 111d via the water supply tube 175.

The air supply nozzle 111e is an opening that communicates with the air supply tube 176. A gas in a tank installed in the vicinity of the control device 700 is sent out from the air supply nozzle 111e via the air supply tube 176.

The suction nozzle 111f is an opening that communicates with the suction tube 177. A tank installed in the vicinity of the control device 700 sucks a gas or a liquid from the suction nozzle 111f via the suction tube 177.

### Scope 200

The scope 200 is a unit that observes a lesion or the like and is attached to the distal end portion 111. The scope 200 may be bendably attached to protrude from the distal end portion 111 to the distal end side A1. The scope 200 includes an imaging unit 201 and an illumination unit 202.

The imaging unit 201 includes a stereo lens and an imaging element such as a CMOS and images an imaging subject. An imaging signal is sent to the video control device 600 via the imaging cable 173.

The illumination unit 202 is connected to a light guide 174 that guides illumination light and emits illumination light illuminating an imaging subject.

In the electric endoscope system 1000, the whole insertion manipulator 100 may be considered as an “endoscope.” In addition, the scope 200, the imaging cable 173, and the light guide 174 may be considered as an “endoscope.”

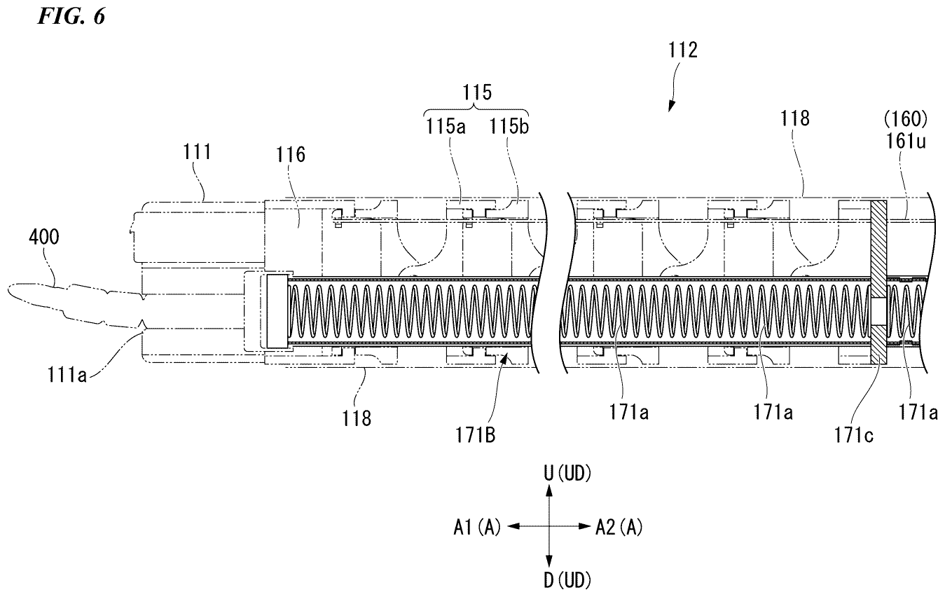

FIG. 6 is diagram illustrating the bending portion 112.

The bending portion 112 includes a plurality of joint rings (also referred to as bending pieces) 115, distal end portions 116 connected to the distal ends of the plurality of joint rings 115, and an outer sheath 118. The plurality of joint rings 115 are connected in the longitudinal direction A in the outer sheath 118. The joint ring 115 at the distal end is connected to the distal end portion 111. In the bending portion 112 illustrated in FIG. 3, the outer sheath 118 is not illustrated.

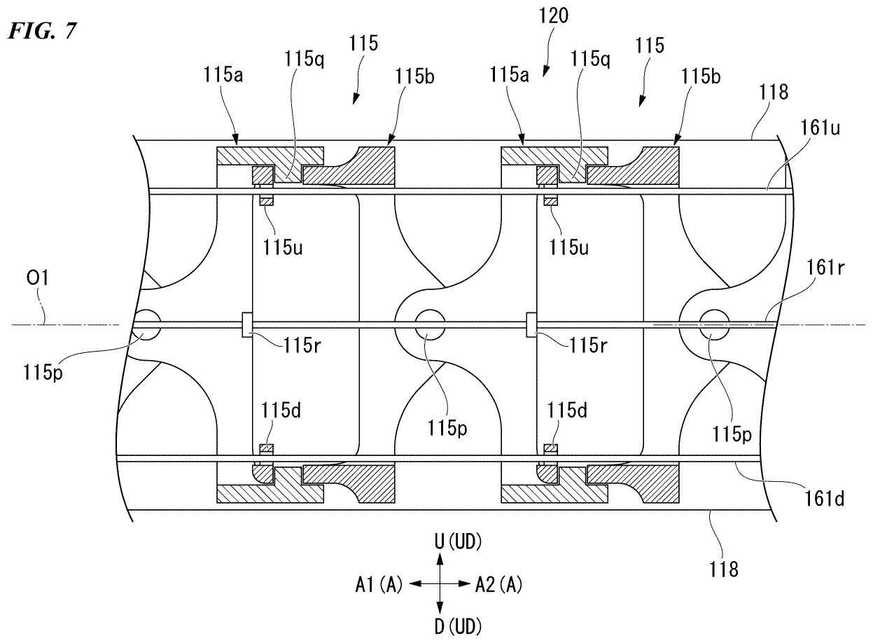

FIG. 7 is a diagram illustrating joint rings 115.

A joint ring 115 is a short cylindrical member formed of metal. A plurality of joint rings 115 are connected such that internal spaces of the neighboring joint rings 115 form a continuous space.

Each joint ring 115 includes a first joint ring 115a on the distal end side and a second joint ring 115b on the proximal side. The first joint ring 115a and the second joint ring 115b are connected by a first rotary pin 115p to be rotatable in a vertical direction (also referred to as an “UD direction”) perpendicular to the longitudinal direction A.

In the neighboring joint rings 115, the second joint ring 115b of the joint ring 115 on the distal end side and the first joint ring 115a of the joint ring 115 on the proximal side are connected by a second rotary pin 115q to be rotatable in a lateral direction (also referred to as an “LR direction”) perpendicular to the longitudinal direction A and the UD direction.

The first joint rings 115a and the second joint rings 115b are alternately connected by the first rotary pin 115p and the second rotary pin 115q, and the bending portion 112 is bendable in a desired direction.

An upper wire guide 115u and a lower wire guide 115d are formed on the inner circumferential surface of the second joint ring 115b. The upper wire guide 115u and the lower wire guide 115d are disposed at both ends in the UD direction with the center axis O1 in the longitudinal direction A interposed therebetween. A left wire guide 115l and a right wire guide 115r are formed in the inner circumferential surface of the first joint ring 115a. The left wire guide 115l and the right wire guide 115r are disposed at both ends in the LR direction with the center axis O1 in the longitudinal direction A interposed therebetween.

The upper wire guide 115u, the lower wire guide 115d, the left wire guide 115l, and the right wire guide 115r are formed such that through-holes into which the bending wire 160 is inserted are parallel to the longitudinal direction A.

The bending wire 160 is a wire for bending the bending portion 112. The bending wire 160 extends to the attachment portion 150 via the internal path 101. The bending wire 160 includes an upper bending wire 161u, a lower bending wire 161d, a left bending wire 1611 (see FIG. 3), and a right bending wire 161r.

The upper bending wire 161u and the lower bending wire 161d are wires for bending the bending portion 112 in the UD direction. The upper bending wire 161u is inserted into the upper wire guide 115u. The lower bending wire 161d is inserted into the lower wire guide 115d.

The left bending wire 161l and the right bending wire 161r are wires for bending the bending portion 112 in the LR direction. The left bending wire 161l is inserted into the left wire guide 115l. The right bending wire 161r is inserted into the right wire guide 115r.

The distal end of the bending wire 160 is fixed to the distal end portion 116 of the bending portion 112. The bending portion 112 is bendable in a desired direction by attracting or relaxing the bending wires 160 (the upper bending wire 161u, the lower bending wire 161d, the left bending wire 161l, and the right bending wire 161r).

The bending wire 160 and the built-in elements 170 (the first channel tube 171, the second channel tube 172, the imaging cable 173, the light guide 174, the water supply tube 175, the air supply tube 176, and the suction tube 177) are inserted into the internal path 101 formed in the bending portion 112. In FIG. 6, built-in elements 170 other than the first channel tube 171 are not illustrated.

The flexible portion 119 is a tubular member which is longitudinal and flexible. The flexible portion 119 includes an outer sheath 118 which is disposed at the outermost. A distal end of the flexible portion 119 is connected to the bending portion 112. The bending wire 160 and the built-in elements 170 (the first channel tube 171, the second channel tube 172, the imaging cable 173, the light guide 174, the water supply tube 175, the air supply tube 176, and the suction tube 177) are inserted into the internal path 101 formed in the flexible portion 119.

The attachment portion 150 is provided at a proximal end of the flexible portion 119 as illustrated in FIG. 1. The attachment portion 150 is attached to the drive device 500 and the video control device 600.

### First Channel Tube 171

As illustrated in FIG. 5, the first channel tube 171 is a tube including a first treatment tool lumen 171r with a large diameter. The second channel tube 172 is a tube including a second treatment tool lumen 172r. The inner diameter D1 of the first treatment tool lumen 171r is larger than the inner diameter D2 of the second treatment tool lumen 172r. Specifically, the inner diameter D1 of the first treatment tool lumen 171r is three to five time the inner diameter D2 of the second treatment tool lumen 172r. As illustrated in FIG. 3, two treatment manipulators 400 can be inserted into the first treatment tool lumen 171r.

The inner diameter D12 of the first treatment tool lumen 171r is equal to or greater than ½ times the outer diameter of the outer sheath 118. In an existing endoscope (for example, a nasal endoscope) in which the inner diameter of a treatment tool channel is relatively large, the outer diameter is about 6 mm and the inner diameter of the treatment tool channel is about 2.4 mm (about ⅖ times the outer diameter). In the insertion manipulator 100, the inner diameter D1 of the first treatment tool lumen 171r with respect to the outer diameter of the outer sheath 118 is larger than that in the existing endoscope. Accordingly, a large treatment manipulator 400 can be inserted into the first treatment tool lumen 171r, and a range of a manual operation is widened.

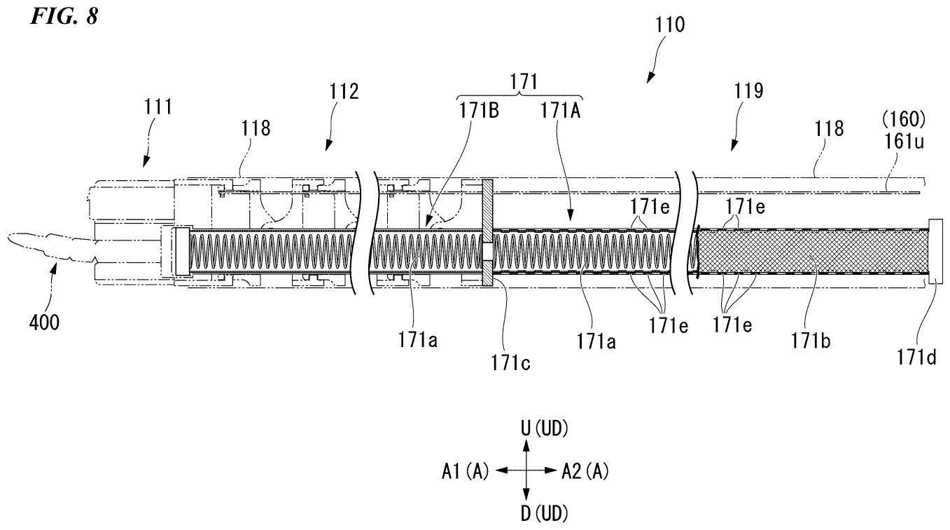

FIG. 8 is a diagram illustrating the first channel tube 171. In FIG. 8, the built-in elements 170 other than the first channel tube 171 are not illustrated.

The first channel tube 171 includes a proximal end channel tube 171A that is disposed in the flexible portion 119 and a distal end channel tube 171B that is disposed in the bending portion 112. The proximal end channel tube 171A and the distal end channel tube 171B are connected to form the first treatment tool lumen 171r.

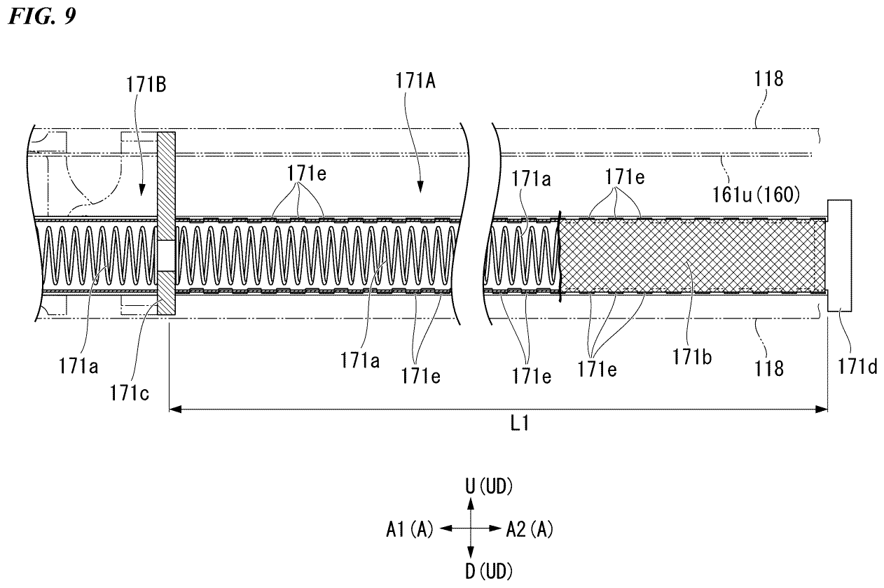

FIG. 9 is a sectional view of the proximal end channel tube 171A.

The proximal end channel tube 171A includes a coil sheath 171a, a blade tube 171b, a first fixed portion 171c, a second fixed portion 171d, and a regulating wire 171e.

The coil sheath 171a is a coil sheath that is flexibly bendable and has excellent kink-resistance. The coil sheath 171a forms the first treatment tool lumen 171r with a large diameter into which the treatment manipulator 400 is inserted.

The blade tube 171b is a tube in which metal strands, resin strands, or the like are woven in a blade shape and is disposed outside of the coil sheath 171a. It is preferable that the blade tube 171b be disposed to be in contact with an outer circumferential surface of the coil sheath 171a.

The proximal end channel tube 171A is a tube with a double structure of the coil sheath 171a and the blade tube 171b. Accordingly, the proximal end channel tube 171A also has excellent torque transmissivity based on the blade tube 171b while maintaining excellent kink-resistance based on the coil sheath 171a. By fitting the coil sheath 171a and the blade tube 171b, a frictional force between the coil sheath 171a and the blade tube 171b is further enhanced, and the torque transmissivity is further enhanced.

The coil sheath 171a is fixed to the blade tube 171b at a first fixed portion 171c on the distal end side A1 in the axial direction A and a second fixed portion 171d on the proximal side A2 with respect to the first fixed portion 171c.

The regulating wire 171e is connected to the first fixed portion 171c and the second fixed portion 171d and regulates a distance L1 in the axial direction A between the first fixed portion 171c and the second fixed portion 171d. It is preferable that a plurality of regulating wires 171e be provided. The regulating wire 171e is a wire with high rigidity and is, for example, an NiTi wire.

Since the distance L1 in the axial direction A between the first fixed portion 171c and the second fixed portion 171d is regulated by the regulating wire 171e, it is possible to prevent a decrease in torque transmissivity or a decrease in pushability due to expansion and contraction of the coil sheath 171a and the blade tube 171b.

The regulating wire 171e is provided to creep alternately inside and outside of the blade tube 171b. Accordingly, the regulating wire 171e can appropriately regulate the distance L1 in the axial direction A between the first fixed portion 171c and the second fixed portion 171d regardless of a bent shape of the insertion section 110.

The coil sheath 171a may be fixed to the blade tube 171b by the first fixed portion 171c and the second fixed portion 171d in a state in which the coil sheath is compressed in the axial direction A. In this case, the coil sheath 171a has a biasing force for separating the first fixed portion 171c and the second fixed portion 171d in the axial direction A. A tension which is an attraction in the axial direction A acts on the blade tube 171b. As a result, it is possible to prevent a decrease in torque transmissivity or a decrease in pushability due to contraction of the coil sheath 171a and the blade tube 171b. When this effect is not enough, the regulating wire 171e is not necessarily required.

As described above, the proximal end channel tube 171A includes the first treatment tool lumen 171r with a large diameter, but has excellent kink-resistance, excellent torque transmissivity, and excellent pushability. Accordingly, even when the outer sheath 118 which is the outermost sheath of the flexible portion 119 is thin, the flexible portion 119 can maintain excellent kink-resistance, excellent torque transmissivity, and excellent pushability.

The distal end channel tube 171B may have the same configuration as the proximal end channel tube 171A. However, the bending portion 112 in which the distal end channel tube 171B is provided includes the joint rings 115 with high rigidity and thus has excellent torque transmissivity and pushability. Accordingly, the distal end channel tube 171B has only to include the coil sheath 171a forming the first treatment tool lumen 171r and may not include the blade tube 171b and the regulating wire 171e.

Since the outer sheath 118 can be formed of a thin film as described above, it is possible to achieve a decrease in diameter of the insertion manipulator 100. However, when the first channel tube 171 does not have sufficient kink-resistance, torque transmissivity, or pushability, the outer sheath 118 may be a multi-layered tube including an outer coil sheath into which the first channel tube 171 is inserted and an outer blade tube which is disposed outside of the outer coil sheath.

### Drive Device 500

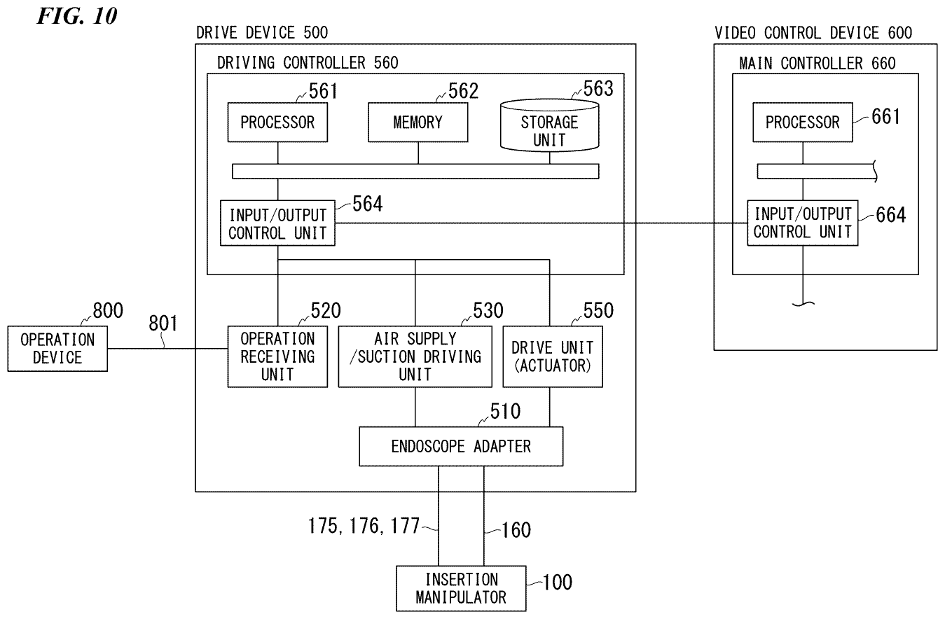

FIG. 10 is a functional block diagram illustrating the drive device 500.

The drive device 500 includes an endoscope adapter 510, an operation receiving unit 520, an air supply/suction driving unit 530, a drive unit 550, and a drive controller 560.

The endoscope adapter 510 is an adapter to which the insertion manipulator 100 is detachably connected. The endoscope adapter 510 connects the bending wire 160, the suction tube 177, the water supply tube 175, and the air supply tube 176 to the drive device 500.

The operation receiving unit 520 receives an operation input from the operation device 800 via the operation cable 801. When the operation device 800 and the drive device 500 communicate in a wireless communication manner instead of a wired communication manner, the operation receiving unit 520 includes a known wireless receiver module.

The air supply/suction driving unit 530 is connected to the suction tube 177, the water supply tube 175, and the air supply tube 176 via the endoscope adapter 510. The air supply/suction driving unit 530 includes a pump and supplies a liquid to the water supply tube 175. The air supply/suction driving unit 530 supplies air to the air supply tube 176. The air supply/suction driving unit 530 sucks air from the suction tube 177.

The drive unit (actuator) 550 is connected to the bending wire 160 of the insertion manipulator 100 via the endoscope adapter 510. The drive unit 550 includes a drive unit and an encoder which are not illustrated. The drive unit attracts or relaxes the bending wire 160 using a pulley or the like. The encoder detects an amount of attraction of the bending wire 160. The detection result of the encoder is acquired by the drive controller 560 of the drive device 500.

The drive unit (actuator) 550 may be connected to the treatment manipulator 400 to drive the treatment manipulator 400.

The drive controller 560 controls the drive device 500 as a whole. The drive controller 560 acquires the operation input received by the operation receiving unit 520. The drive controller 560 controls the air supply/suction driving unit 530 and the drive unit 550 on the basis of the acquired operation input.

The drive controller 560 is a program-executable computer including a processor 561, a memory 562, a storage unit 563 storing a program and data, and an input/output control unit 564. The functions of the drive controller 560 are realized by causing the processor 561 to execute a program. At least some functions of the drive controller 560 may be realized by a dedicated logical circuit.

The drive controller 560 may further include an element in addition to the processor 561, the memory 562, the storage unit 563, and the input/output control unit 564. For example, the drive controller 560 may further include an image computing unit that performs some or all of image processing and image recognition processing. When the image computing unit is additionally provided, the drive controller 560 can perform specific image processing or image recognition processing at a high speed. The image computing unit may be mounted in a separate hardware device connected thereto via a communication line.

### Video Control Device 600

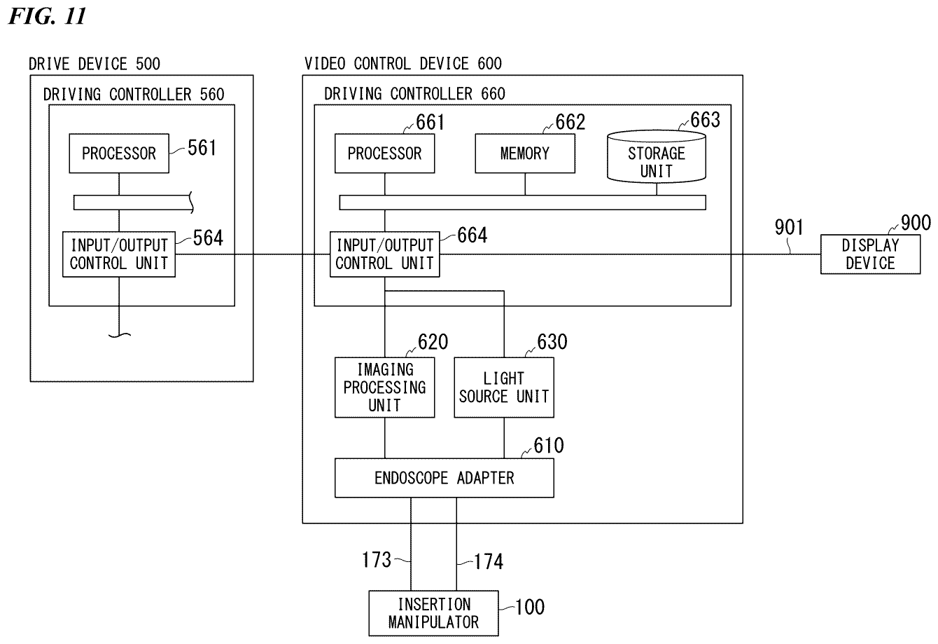

FIG. 11 is a functional block diagram illustrating the video control device 600.

The video control device 600 includes an endoscope adapter 610, an imaging processing unit 620, a light source unit 630, and a main controller 660.

The endoscope adapter 610 is an adapter to which the insertion manipulator 100 is detachably connected. The endoscope adapter 610 connects the imaging cable 173 and the light guide 174 to the video control device 600.

The imaging processing unit 620 is connected to the imaging cable 173. The imaging processing unit 620 converts an imaging signal acquired from the imaging unit 201 of the scope 200 via the imaging cable 173 and the imaging cable 173 to a captured image.

The light source unit 630 is connected to the light guide 174. The light source unit 630 generates illumination light which is applied to an imaging subject. The illumination light generated by the light source unit 630 is guided to an illumination unit 202 of the scope 200 via the light guide 174.

The main controller 600 is a program-executable computer including a processor 661, a memory 662, a storage unit 663 that can store a program and data, and an input/output control unit 664. The functions of the main controller 660 are realized by causing the processor 661 to execute a program. As least some functions of the main controller 660 may be realized by a dedicated logical circuit.

The main controller 660 can perform image processing on a captured image acquired by the imaging processing unit 620. The main controller 660 can generate a GUI image or a CG image for providing information to an operator S. The main controller 660 can display the captured image, the GUI image, or the CG image on the display device 900.

The main controller 660 is not limited to an integrated hardware device. For example, the main controller 660 may be constituted by separating a part thereof as a separate hardware device and connecting the separated hardware device thereto via a communication line. For example, the main controller 660 may be a cloud system that connects the separated storage units 663 via a communication line.

The main controller 660 may further include an element in addition to the processor 661, the memory 662, the storage unit 663, and the input/output control unit 664. For example, the main controller 660 may further include an image computing unit that performs some or all of image processing or image recognition processing which is performed by the processor 661. Since the image computing unit is further provided, the main controller 660 can perform specific image processing or image recognition processing at a high speed. The image computing unit may be mounted in a separate hardware device connected thereto via a communication line.

### Operation of Electric Endoscope System 1000

Operations of the electric endoscope system 1000 according to the present embodiment will be described below. Specifically, manual operations of observing and treating a lesion formed in a wall of a large intestine using the electric endoscope system 1000 will be described.

An operator S inserts the insertion section 110 of the insertion manipulator 100 into the large intestine via an anal passage of a patient from its distal end. The operator S operates the operation device 800 while observing the captured image displayed on the display device 900 such that the insertion section 110 is moved to cause the distal end portion 111 to approach a lesion. The operator S operates the operation device 800 to bend the bending portion 112 according to necessity.

Since the insertion manipulator 100 has excellent kink-resistance, excellent torque transmissivity, and excellent pushability, the operator S can easily operate the insertion manipulator 100.

The operator S inserts the treatment manipulator 400 into the first channel tube 171 or the second channel tube 172. The operator S operates the operation device 800 while observing the captured image displayed on the display device 900 such that the treatment manipulator 400 is operated to treat the lesion.

Since the insertion manipulator 100 includes the first treatment tool lumen 171r with a large diameter, the operator S can appropriately perform treatment using the large treatment manipulator 400.

After treating the lesion, the operator S removes the insertion manipulator 100 and the treatment manipulator 400 and ends the manual operation.

With the electric endoscope system 1000 according to the present embodiment, it is possible to more efficiently perform observation or treatment. Since the insertion manipulator 100 has excellent kink-resistance, excellent torque transmissivity, and excellent pushability, the operator S can easily operate the insertion manipulator 100. Since the insertion manipulator 100 includes the first treatment tool lumen 171r with a large diameter, the operator S can appropriately perform treatment using the large treatment manipulator 400.

While the first embodiment of the present invention has been described above in details with reference to the drawings, a specific configuration is not limited to this embodiment and includes a change in design without departing from the gist of the present invention. Elements described in the aforementioned embodiment and modified examples can be appropriately combined into a configuration.

### Modified Example 1-1

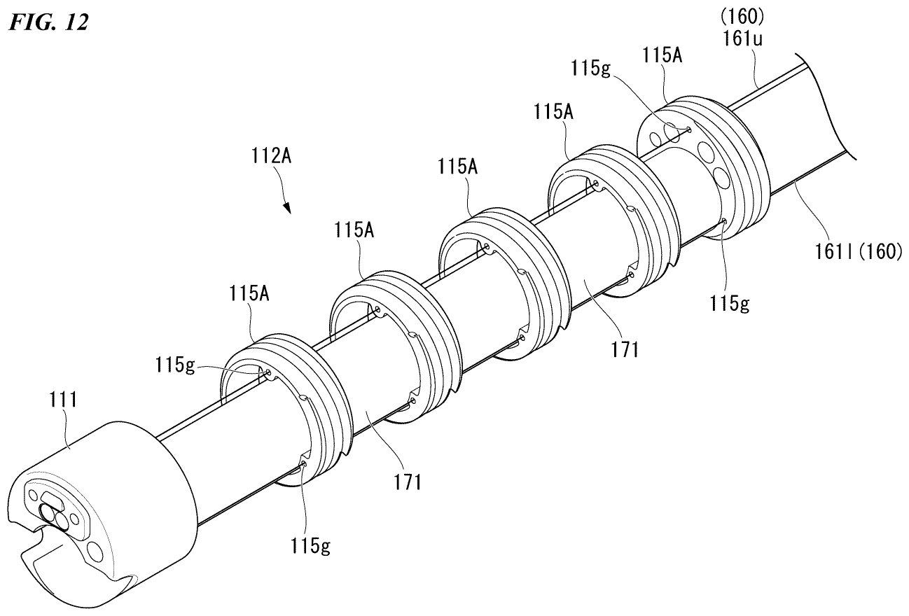

FIG. 12 is a diagram illustrating a bending portion 112A which is a modified example of the bending portion 112. In FIG. 12, the built-in elements 170 other than the first channel tube 171 are not illustrated.

The bending portion 112 does not include a plurality of joint rings 115 but includes a plurality of ring members 115A. The plurality of ring members 115A are arranged in the axial direction A. The built-in elements 170 including the first channel tube 171 are inserted into the plurality of ring members 115A. The bending wire 160 is inserted into a wire guide 115g formed in the ring members 115A.

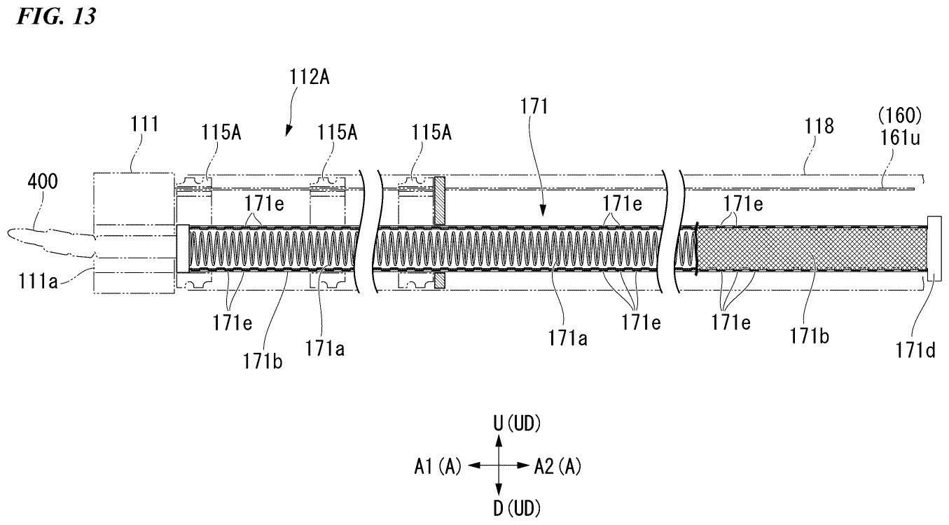

FIG. 13 is a diagram illustrating the first channel tube 171 which is inserted into the bending portion 112A.

The bending portion 112A has lower torque transmissivity or lower pushability in comparison with the bending portion 112 in which the joint rings 115 are connected. Accordingly, a distal end portion of the first channel tube 171 into which the bending portion 112A is inserted is preferably a tube with a double structure of the coil sheath 171a and the blade tube 171b. The distal end portion of the first channel tube 171 into which the bending portion 112A is inserted preferably includes a regulating wire 171e for regulating a distance between the ends.

FIG. 14 is a diagram illustrating an insertion section 110A which is a modified example of the insertion section 110.

The insertion section 110A includes an expanding and contracting portion 117 in addition to a distal end portion 111, a bending portion 112, and a flexible portion 119. The expanding and contracting portion 117 is a member that connects the distal end portion 116 of the bending portion 112 to the distal end portion 111 and is driven by the drive unit (actuator) 550 to expand and contract in the longitudinal direction A. An outer circumferential portion of the expanding and contracting portion 117 is formed in a bellows shape. The operator S can accurately control a position of the distal end portion 111 in the longitudinal direction A with expansion and contraction of the expanding and contracting portion 117.

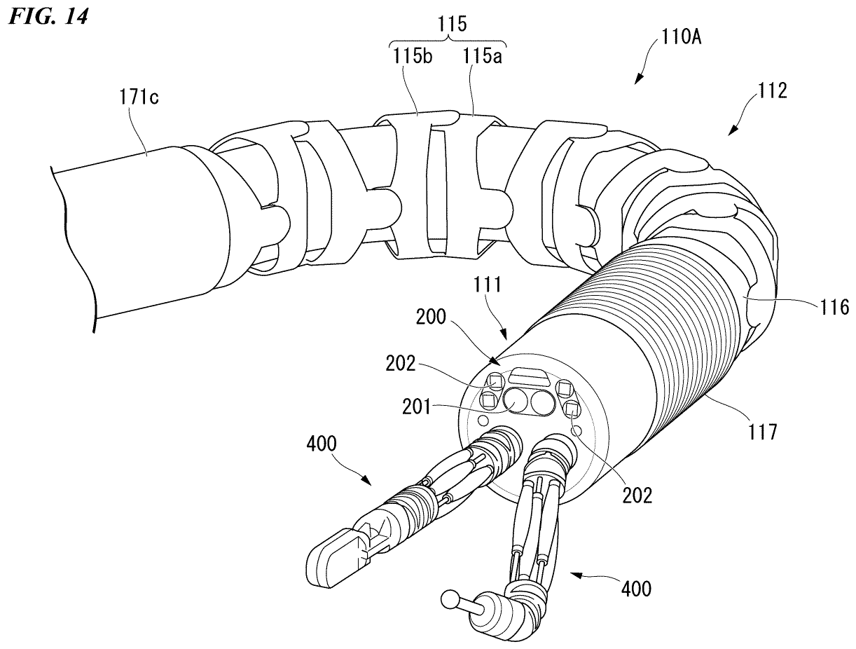

FIG. 15 is a diagram illustrating a spiral tube 180.

The operator may insert the insertion section 110 into a spiral tube 180 which is separate from the insertion section 110. The spiral tube 180 is a tube in which a fin 181 is wound in a spiral shape on an outer circumference thereof. The spiral tube 180 is driven by the drive unit (actuator) 550 to be rotatable about a rotation axis extending in the longitudinal direction A. The operator can cause the spiral tube 180 and the insertion section 110 to move forward and rearward in the lumen by rotating the spiral tube 180. For example, the operator can easily insert the distal end portion 111 of the insertion section 110 to a deep region of a large intestine.

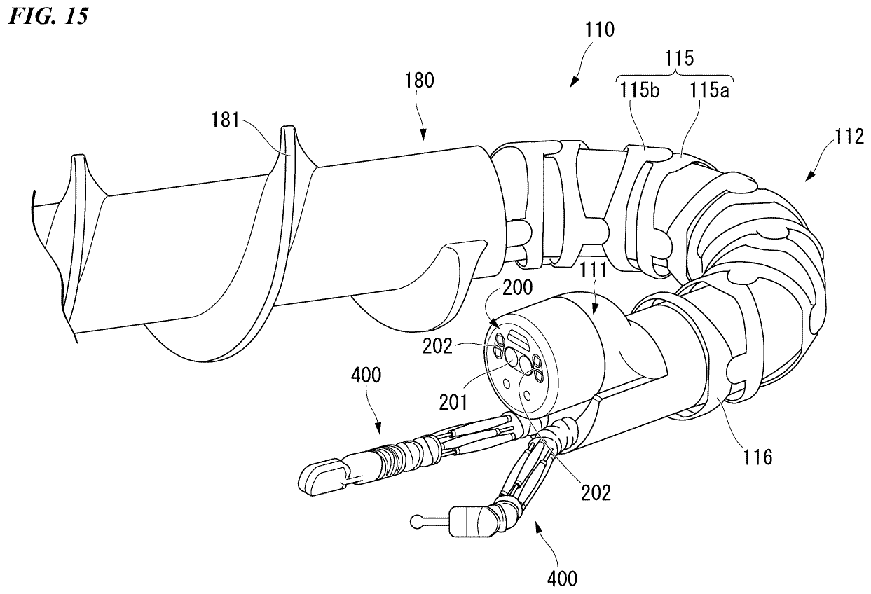

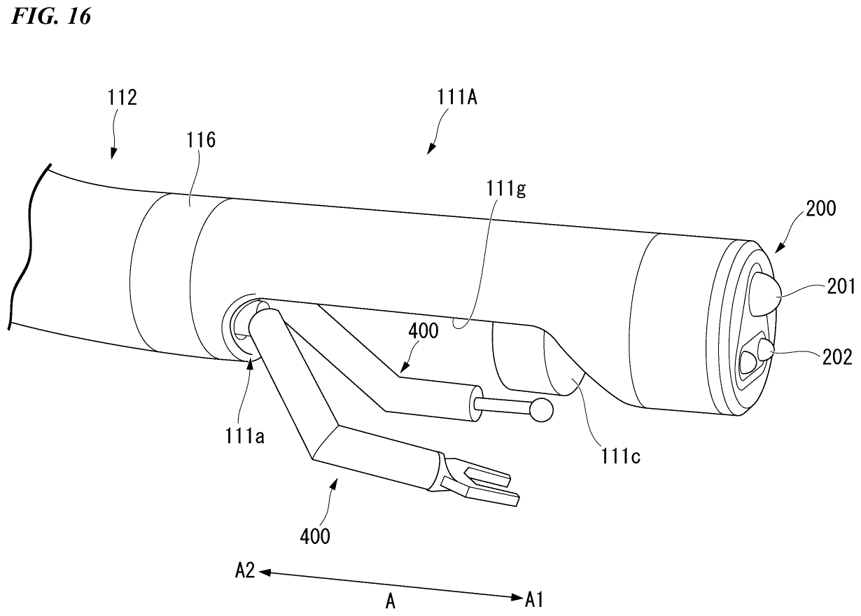

FIG. 16 is a diagram illustrating a distal end portion 111A which is a modified example of the distal end portion 111.

The distal end portion 111A includes a cutout portion 111g in which a part of a cylindrical body is cut out. The cutout portion 111g extends in the longitudinal direction A. A first opening 111a of the distal end portion 111A is provided on the proximal side A2 of the cutout portion 111g. The first opening 111a is open to the distal end side A1. The scope 200 is attached to the distal end of the distal end portion 111A. The distal end portion 111A includes a treatment camera 111c in addition to the scope 200 which is an insertion camera. The treatment camera 111c is provided on the distal end side A1 of the cutout portion 111g. The first opening 111a and the treatment camera 111c are provided to face each other. The treatment camera 111c can image a situation in which the treatment manipulator 400 protruding from the first opening 111a teats a lesion. Since the direction which the treatment camera 111c faces and the direction in which the treatment manipulator 400 protrudes are opposite, the operator can perform treatment while normally viewing the distal end of the treatment manipulator 400.

### Second Embodiment

A hardness changing device 300 according to a second embodiment of the present disclosure will be described below with reference to FIGS. 17 to 25. In the following description, the same constituents as in the above description will be referred to by the same reference signs, and repeated description thereof will be omitted.

FIG. 17 is a diagram a hardness changing device 300 which is inserted into the bending portion 112.

The hardness changing device (rigidiser, introducer) 300 is a device including a hardness changing portion 310 into which the first treatment tool lumen 171r can be inserted. In FIG. 17, the hardness changing device 300 is inserted into the first treatment tool lumen 171r. In FIG. 17, the first channel tube 171 and the second channel tube 172 are not illustrated.

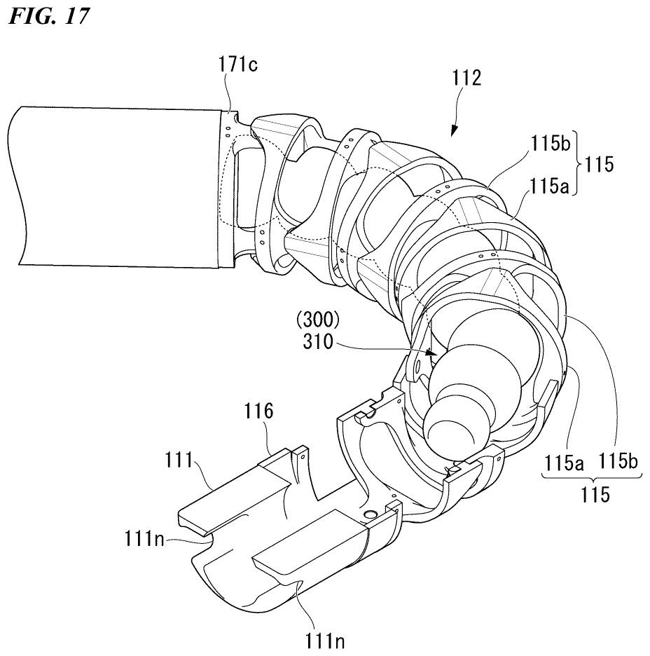

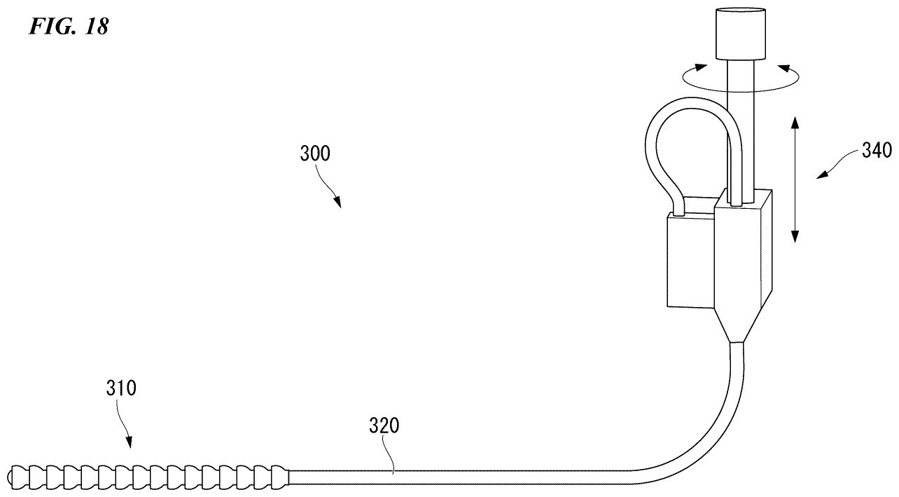

FIG. 18 is a diagram illustrating the entire configuration of the hardness changing device 300.

The hardness changing device 300 includes a hardness changing portion 310, an insertion section 320, and a drive unit 340.

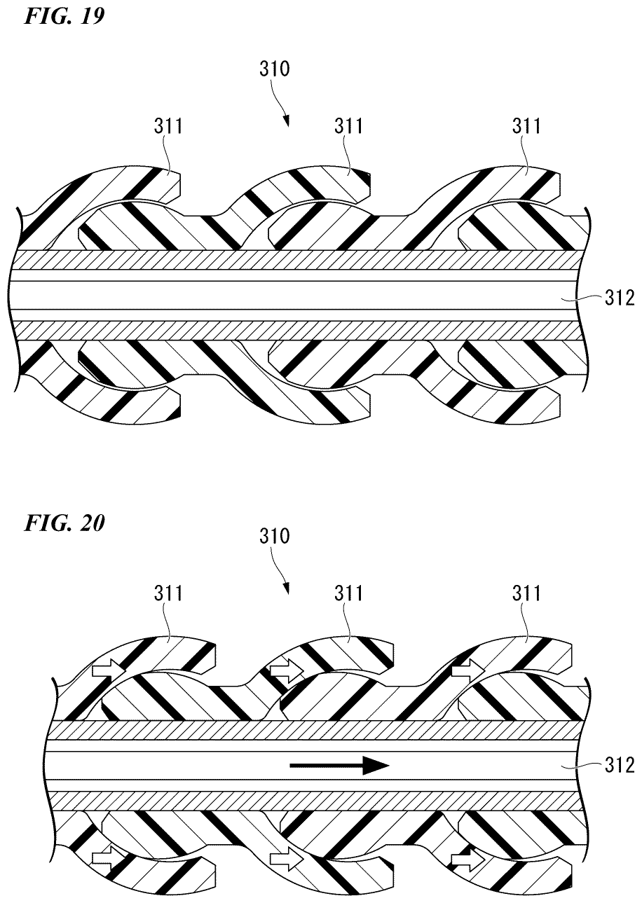

FIGS. 19 and 20 are diagrams illustrating the hardness changing portion 310.

The hardness changing portion 310 is a long member which can be inserted into the first treatment tool lumen 171r and in which hardness changes by performing a predetermined operation thereon. The hardness changing portion 310 includes a plurality of spinal portions 311 and a wire 312. Each spinal portion 311 is formed in a bowl shape. The plurality of spinal portions 311 are connected in a longitudinal axis direction while overlapping each other. The wire 312 is inserted into the plurality of spinal portions 311 and is fixed to a spinal portion at the distal end. When the wire 312 is attracted to the proximal side as illustrated in FIG. 20, neighboring spinal portions 311 come into close contact, and thus a frictional force is enhanced and the shape of the hardness changing portion 310 is fixed. By loosening the wire 312, the shape of the hardness changing portion 310 changes.

The insertion section 320 is a long member that can be inserted into the first treatment tool lumen 171r and has flexibility. The insertion section 320 is provided on the proximal side of the hardness changing portion 310.

The drive unit 340 is provided on the proximal side of the insertion section 320. The drive unit 340 causes the insertion section 320 to move forward and rearward in the longitudinal axis direction and causes the insertion section 320 to rotate about the longitudinal axis. The drive unit 340 may be incorporated into the drive device 500.

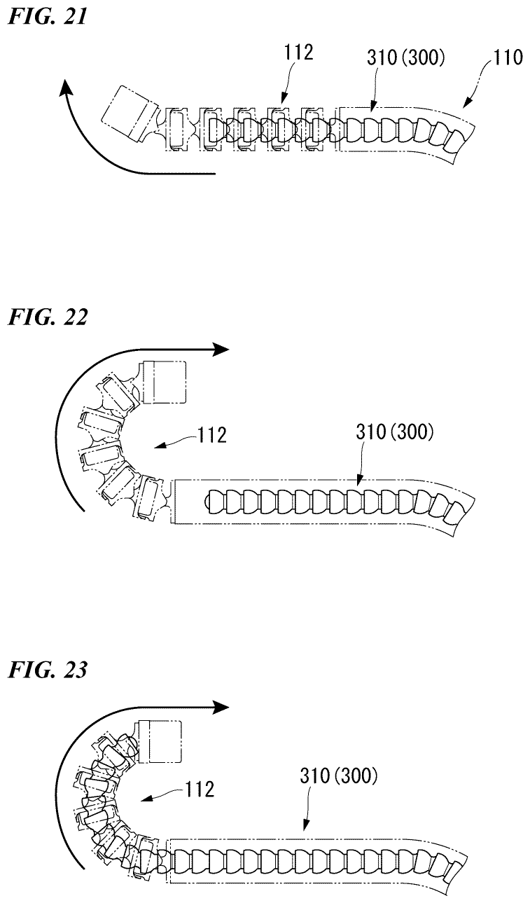

FIGS. 21 to 25 are diagrams illustrating operations of the hardness changing portion 310.

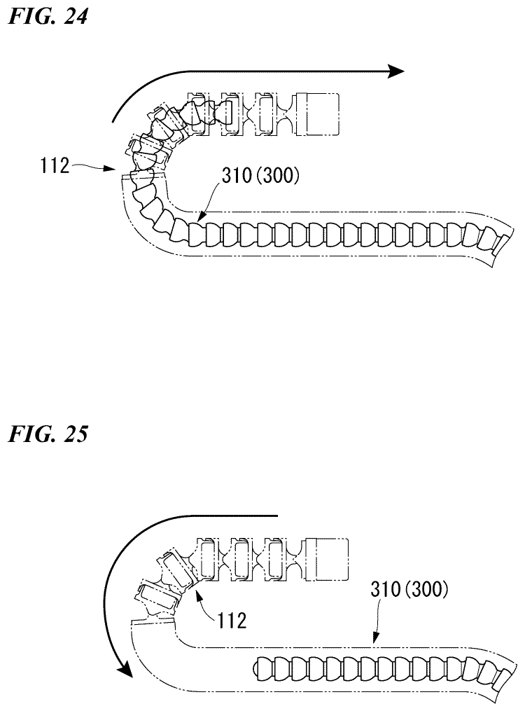

As illustrated in FIG. 21, the operator S inserts the distal end of the insertion section 110 of the insertion manipulator 100 into a large intestine of a patient via an anal passage. As illustrated in FIG. 22, the operator S disposes the bending portion 112 in a part of the large intestine which is greatly bent. As illustrated in FIG. 23, the operator S causes the hardness changing portion 310 with a variable shape to move forward and locates the hardness changing portion 310 in the bending portion 112. The operator S fixes the shape of the hardness changing portion 310 inserted into the bending portion 112. As illustrated in FIG. 24, the operator S causes the insertion section 110 to move forward. The bending portion 112 moves forward along the hardness changing portion 310 with a fixed shape. The insertion manipulator 100 can smoothly pass through the part of the large intestine which is greatly bent. When the hardness changing portion 310 is not necessary, the operator S opens the wire of the hardness changing portion 310 and removes the hardness changing portion 310 from the bending portion 112 in a state in which it is softened as illustrated in FIG. 25.

With the hardness changing device 300 according to the present embodiment, it is possible to more efficiently perform observation or treatment. The operator S can smoothly insert the insertion manipulator 100 to a target position using the hardness changing device 300.

While the second embodiment of the present invention has been described above in details with reference to the drawings, a specific configuration is not limited to this embodiment and includes a change in design without departing from the gist of the present invention. Elements described in the aforementioned embodiment and modified examples can be appropriately combined into a configuration.

### Modified Example 2-1

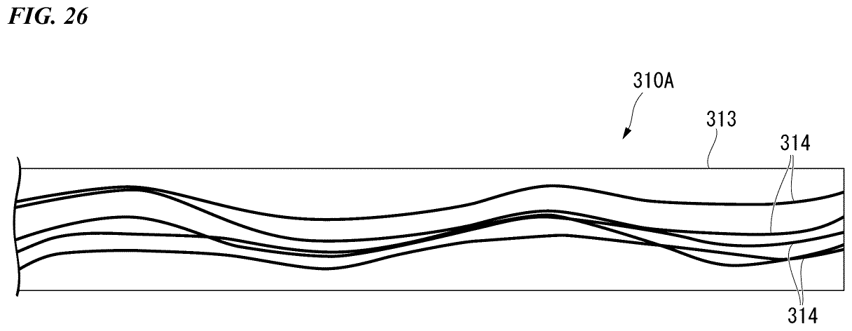

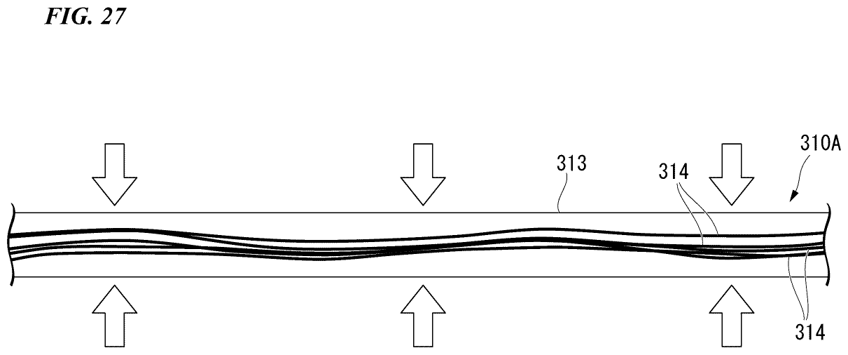

FIGS. 26 and 27 are diagrams illustrating a hardness changing portion 310A which is a modified example of the hardness changing portion 310. The hardness changing portion 310A includes a tube 313 and a plurality of wire members 314 which are inserted into the tube 313. Each wire member 314 is a metal strand or a resin strand. As illustrated in FIG. 27, by making the internal space of the tube 313 have a negative pressure through suction and brining the plurality of wire members 314 into contact with each other, relative movement of the wire members 314 is curbed due to a frictional force between the plurality of wire members 314 and between the wire member 314 and the tube. Accordingly, the shape of the hardness changing portion 310A is fixed. Since the frictional force is increased by roughening the surfaces of the wire members 314 to increase contact resistance, it is possible to further enhance hardness of the hardness changing portion 310A with a fixed shape.

### Modified Example 2-2

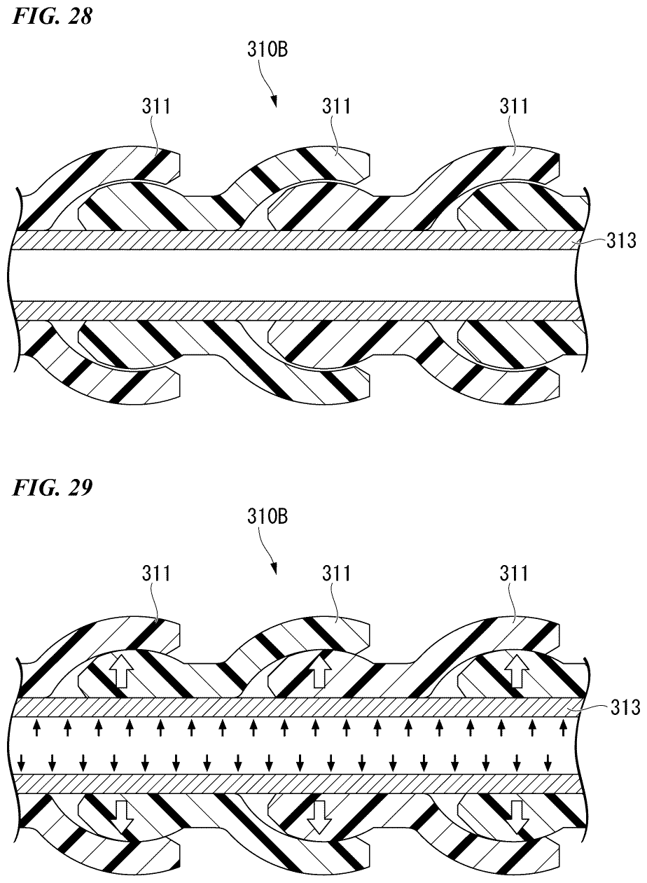

FIGS. 28 and 29 are diagrams illustrating a hardness changing portion 310B which is a modified example of the hardness changing portion 310. The hardness changing portion 310B includes a plurality of spinal portions 311 and a tube 313. The tube 313 is inserted into the plurality of spinal portions 311. As illustrated in FIG. 29, by making the internal space of the tube 313 have a positive pressure through suction such that the tube 313 expands, a frictional force between the neighboring spinal portions 311 is increased, and the shape of the hardness changing portion 310B is fixed. Since the hardness changing portion 310B makes the internal space of the tube 313 have a positive pressure, it is possible to easily increase the frictional force between the plurality of spinal portions 311 and to more strength the hardness changing portion 310B in comparison with a mode in which the internal space of the tube 313 is made to have a negative pressure. In the mode in which the internal space of the tube 313 is made to have a negative pressure, the pressure has only to be reduced by only the atmospheric pressure. However, when the internal space of the tube 313 is made to have a positive pressure, such limitation is removed, and thus it is possible to apply a larger pressure.

### Modified Example 2-3

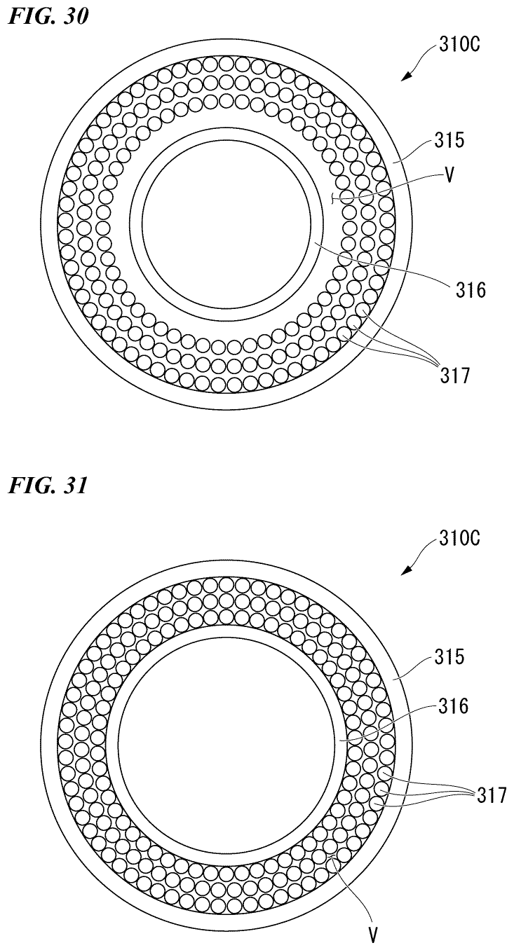

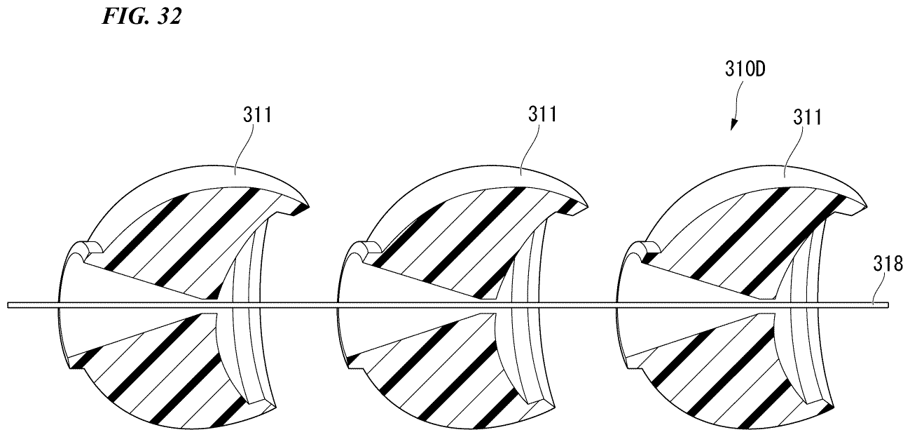

FIGS. 30 and 31 are diagrams illustrating a hardness changing portion 310C which is a modified example of the hardness changing portion 310. The hardness changing portion 310C includes an outer tube 315, an inner tube 316, and a plurality of cables 317. The inner tube 316 is inserted into the inner space of the outer tube 315. The plurality of cables 317 are inserted into a space V interposed between the outer tube 315 and the inner tube 316. As illustrated in FIG. 31, by making the space V have a negative pressure through suction, the plurality of cables 317 come into contact with each other, and the shape of the hardness changing portion 310C is fixed. The space V may be made to have a negative pressure using a hydraulic pressure.

### Modified Example 2-4

FIG. 32 is a diagram illustrating a hardness changing portion 310D which is a modified example of the hardness changing portion 310. The hardness changing portion 310D includes a plurality of spinal portions 311 and a shape memory alloy wire 318. In the hardness changing portion 310D, when the shape memory alloy wire 318 contracts with supply of electric power, the spinal portions 311 come into close contact with each other similarly to the hardness changing portion 310, and thus the hardness changing portion 310D can switch between a state in which the shape is fixed and a state in which the shape is variable.

### Third Embodiment

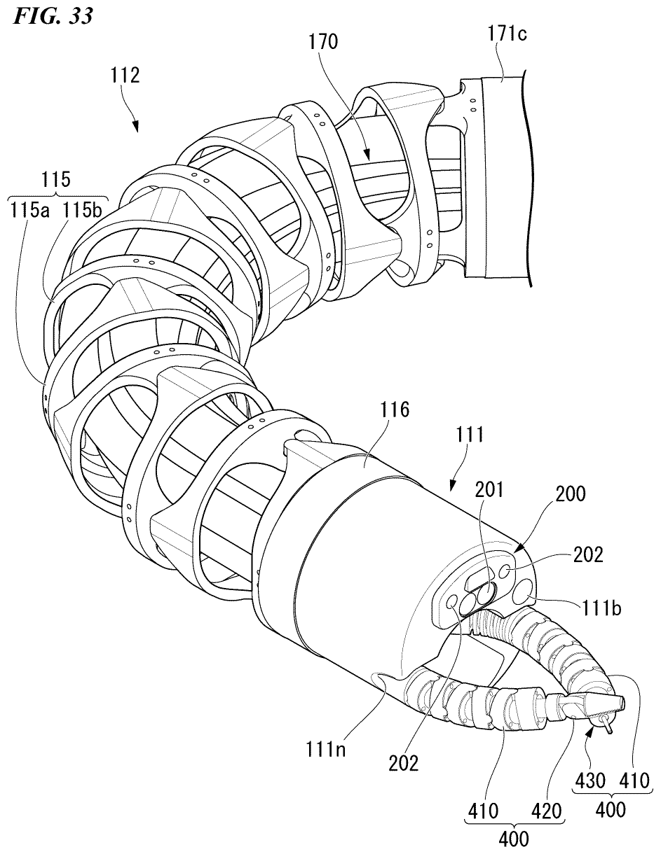

A treatment manipulator 400 according to a third embodiment of the present disclosure will be described below with reference to FIGS. 33 to 40. In the following description, the same constituents as in the above description will be referred to by the same reference signs, and repeated description thereof will be omitted.

FIG. 33 is a diagram illustrating a high-frequency knife 430.

The treatment manipulator 400 includes a bendable treatment tool arm 410 and treatment tools (a forceps 420, a high-frequency knife 430, a local injection needle 440, and a basket 450) which are inserted into the treatment tool arm 410.

The treatment tool arm 410 is a hollow long member. Similarly to the bending portion 112 of the insertion manipulator 100, the treatment tool arm 410 includes a plurality of joint rings (also referred to as bending pieces) 415 and is driven with a wire or the like to be bendable in the vertical direction and the lateral direction.

The treatment tools (such as the forceps 420, the high-frequency knife 430, the local injection needle 440, and the basket 450) can be inserted into the internal space (a lumen or a channel) of the treatment tool arm 410. The treatment tools are manually bendable, but may not have an active bending function.

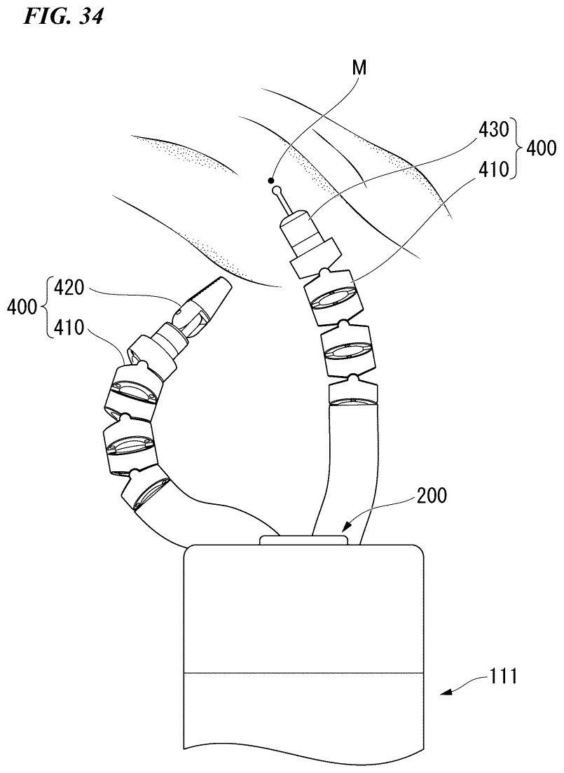

FIG. 34 is a diagram illustrating the high-frequency knife 430 for putting a marking M.

An operator S inserts the high-frequency knife 430 into the treatment tool arm 410 and causes the distal end of the high-frequency knife 430 to protrude from the distal end of the treatment tool arm 410. The operator S locates the distal end of the high-frequency knife 430 at a desired position by bending the treatment tool arm 410. The operator S cauterize biological tissue with the distal end portion of the high-frequency knife 430 and puts a marking M on the biological tissue.

Since the first treatment tool lumen 171r has a large diameter, two treatment tool arms 410 can be inserted thereinto. For example, as illustrated in FIG. 34, the operator S can insert the high-frequency knife 430 into one treatment tool arm 410, insert the forceps 420 into the other treatment tool arm 410, and treat a target part using two treatment tools.

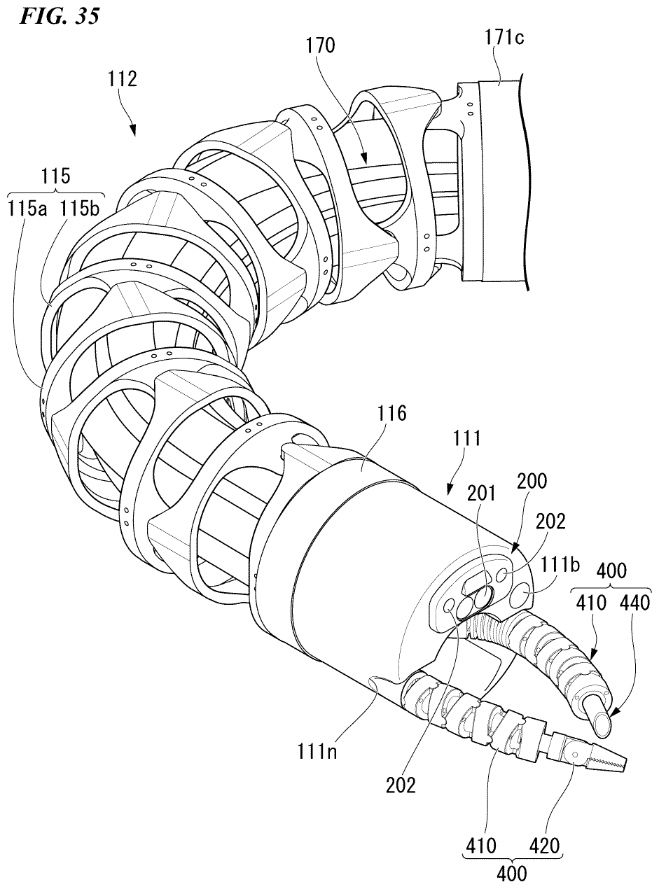

FIG. 35 is a diagram illustrating a local injection needle 440.

The operator S removes the high-frequency knife 430 from the treatment tool arm 410. Then, the operator S inserts the local injection needle 440 into the treatment tool arm 410 and causes the distal end of the local injection needle 440 to protrude from the distal end of the treatment tool arm 410.

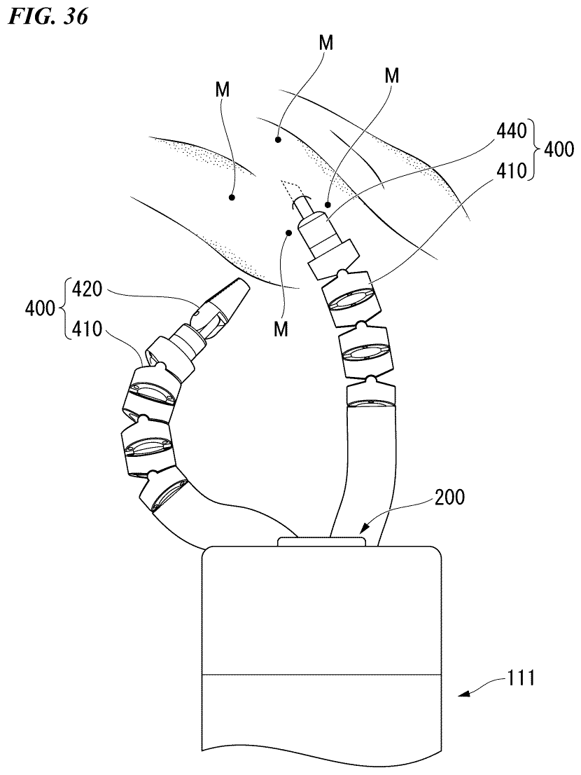

FIG. 36 is a diagram illustrating the local injection needle 440 which locally injects a liquid.

The operator S bends the treatment tool arm 410 and locates the distal end of the local injection needle 440 at a desired position. The operator S pierces the biological tissue with the distal end of the local injection needle 440 and locally injects a liquid.

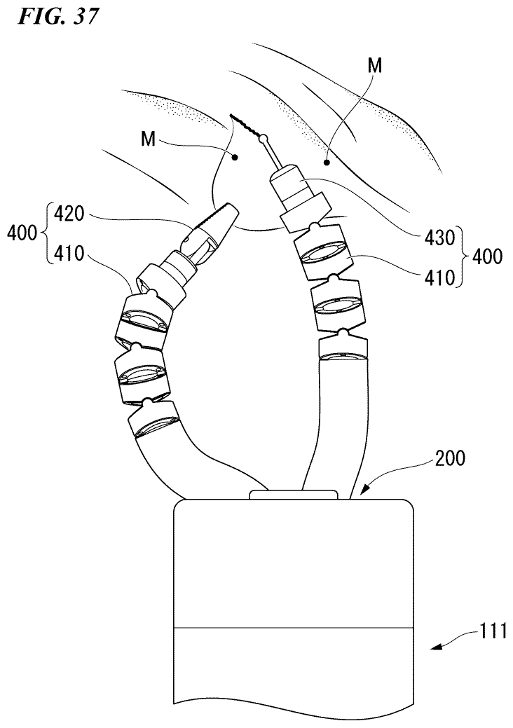

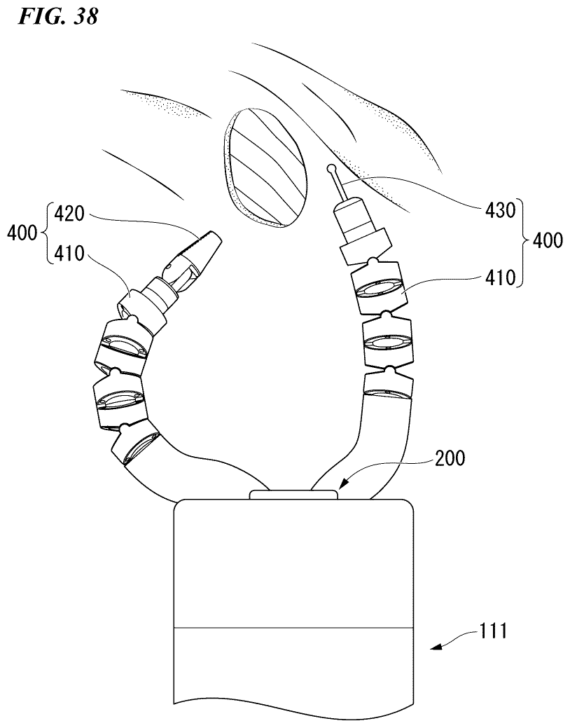

FIGS. 37 and 38 are diagrams illustrating a high-frequency knife 430 for cutting.

The operator S removes the local injection needle 440 from the treatment tool arm 410. Then, the operator S inserts the high-frequency knife 430 into the treatment tool arm 410. The operator S attracts biological tissue using the forceps 420. The operator S cuts a target part of the biological tissue with the high-frequency knife 430 while checking the marking M.

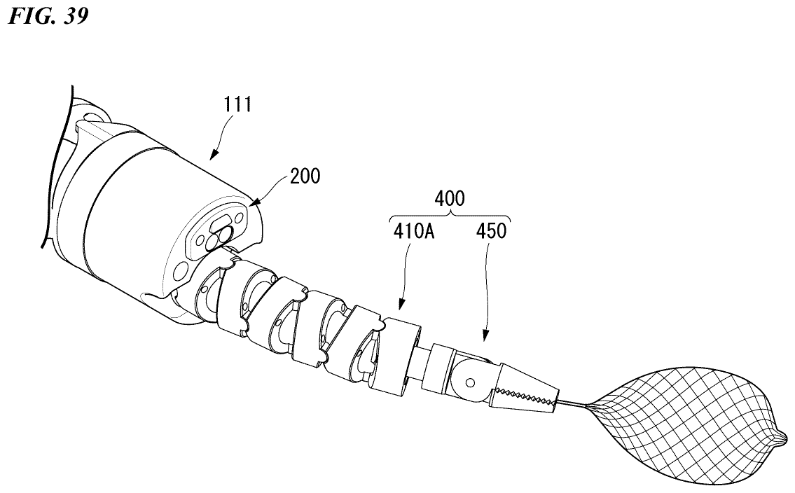

FIG. 39 is a diagram illustrating a basket 450.

The operator S removes the two treatment tool arms 410 along with the treatment tools. Then, the operator S inserts a large-diameter treatment tool arm 410A into the first treatment tool lumen 171r. The operator S inserts the basket 450 into the large-diameter treatment tool arm 410A. The basket 450 has a larger outer diameter than that of the high-frequency knife 430 or the local injection needle 440. The large-diameter treatment tool arm 410A has a larger inner diameter of a hollow portion than that of the treatment tool arm 410 and enables the basket 450 to be inserted thereinto. Only a large basket 450 may be directly inserted into the first treatment tool lumen 171r without using the large-diameter treatment tool arm 410A.

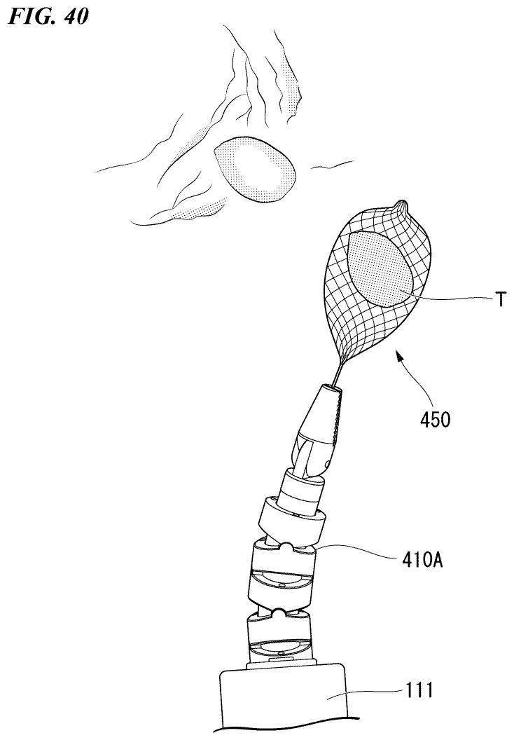

FIG. 40 is a diagram illustrating the basket 450 for recovering a target part T.

The operator S recovers a target part T which has been cut and removed using the basket 450. The operator S can efficiently recover the target part T using the large basket 450. Since the first treatment tool lumen 171r has a large diameter, the large basket 450 larger than a basket used in the related art can be inserted thereinto. Since the first treatment tool lumen 171r has a large diameter, a larger amount of target part T or tissue than that in the related art can be easily recovered at a time without being caught by the first treatment tool lumen 171r. Since the first treatment tool lumen 171r has a large diameter, larger treatment tools than those in the related art in addition to the basket can be inserted thereinto.

With the treatment manipulator 400 according to the present embodiment, it is possible to more efficiently perform observation or treatment. Since the insertion manipulator 100 has the first treatment tool lumen 171r with a large diameter, two treatment manipulators 400 can be simultaneously inserted and used. Since the treatment manipulator 400 with a large diameter can be inserted into the first treatment tool lumen 171r, a multi-functional and high-performance large treatment manipulator 400 can be used.

While the third embodiment of the present invention has been described above in details with reference to the drawings, a specific configuration is not limited to this embodiment and includes a change in design without departing from the gist of the present invention. Elements described in the aforementioned embodiment and modified examples can be appropriately combined into a configuration.

### Fourth Embodiment

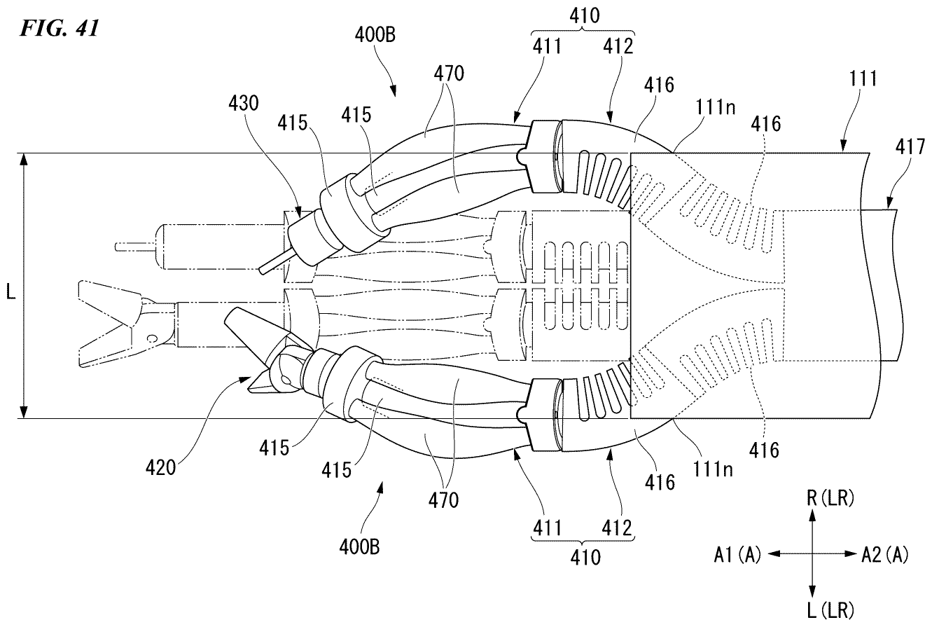

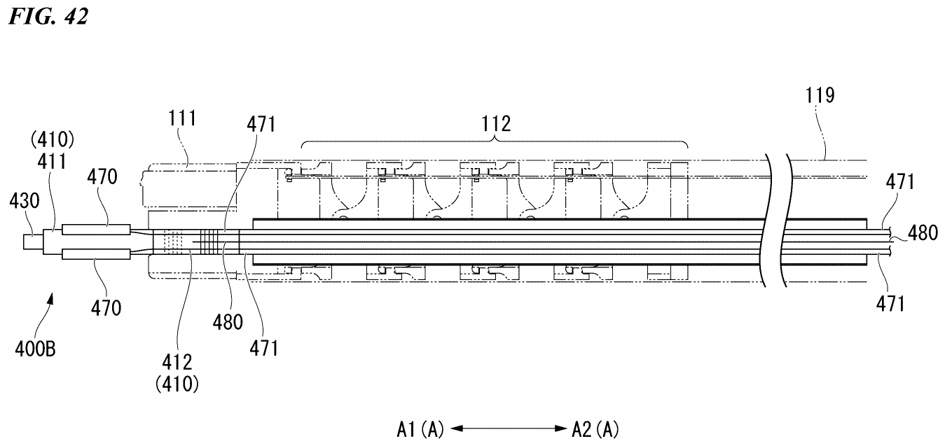

A treatment manipulator 400B according to a fourth embodiment of the present disclosure will be described below with reference to FIGS. 41 to 42. In the following description, the same constituents as in the above description will be referred to by the same reference signs, and repeated description thereof will be omitted.

FIG. 41 is a diagram illustrating a treatment manipulator 400B.

Similarly to the treatment manipulator 400 according to the aforementioned embodiment, the treatment manipulator 400B is a device that is inserted into the first channel tube 171 or the like of the insertion manipulator 100 and is inserted into a lumen of a patient to treat a lesion. The treatment manipulator 400B is driven by the drive unit (actuator) 550 or the like. The treatment manipulator 400B is different from the treatment manipulator 400 in that two treatment tool arms 410 are arranged on a distal end surface of one manipulator flexible portion 417. The treatment manipulator 400B can be more easily inserted in comparison with the treatment manipulator 400.

The treatment manipulator 400B includes a treatment tool (such as a forceps 420, a high-frequency knife 430, a local injection needle 440, or a basket 450), a treatment tool arm 410, an artificial muscle 470, and a wire 480.

The treatment tool arm 410 is a hollow long member. The treatment tool arm 410 includes a first bending portion 411 provided on the distal end side A1 and a second bending portion 412 provided on the proximal side A2. In the use state of the treatment manipulator 400B illustrated in FIG. 41, the first bending portion 411 is located on the distal end side A1 with respect to the distal end surface of the distal end portion 111. A part of the second bending portion 412 protrudes from the distal end portion 111 to the distal end side A1.

The first bending portion 411 includes a plurality of joint rings (also referred to as wrist joints) 415 similarly to the bending portion 112 of the insertion manipulator 100 and can be driven by the artificial muscle 470 such that it is bendable in the vertical direction and the lateral direction.

As illustrated in FIG. 41, the second bending portion 412 includes a shoulder joint 416 that greatly bends the treatment tool arm 410 in the lateral direction (LR direction). The shoulder joint 416 bends the second bending portion 412 in an S-shape when seen in the vertical direction (UD direction). As illustrated in FIG. 41, the distal ends of the second bending portions 412 of two treatment tool arms 410 are bent and separated from each other in the lateral direction. Here, a distance between the center axes at the distal ends of the second bending portions 412 of the two treatment tool arms 410 is defined as a “distance (shoulder width) L.”

When a target part is treated using two treatment tool arms 410, the operator S can secure space enough for treatment and easily treat the target part by appropriately separating the positions of the proximal ends of the two first bending portions 411. Therefore, the operator S greatly bends the second bending portions 412 such that the distance L is appropriately increased. At least a part of the two treatment manipulators 400 can be inserted into cutout portions 111n and spread outward in the lateral direction. Accordingly, the distance L may be larger than the outer diameter of the distal end portion 111 of the insertion manipulator 100.

The artificial muscle 470 constitutes at least a part of a mechanism for bending a treatment tool. The artificial muscle 470 is, for example, a Mckibben artificial muscle. The artificial muscle 470 is attached to the first bending portion 411 and bends the first bending portion 411. In order to bend the first bending portion 411 in the vertical direction and the lateral direction, a plurality of artificial muscles 470 are attached to the first bending portion 411.

In the use state of the treatment manipulator 400B illustrated in FIG. 41, the first bending portion 411 is disposed on the distal end side A1 with respect to the distal end surface of the distal end portion 111. Accordingly, the artificial muscle 470 is also disposed on the distal end side A1 with respect to the distal end surface of the distal end portion 111.

FIG. 42 is a sectional view of the treatment manipulator 400B.

A tube 471 for supplying a fluid for operating the artificial muscle 470 is attached to the artificial muscle 470. The artificial muscle 470 is contracted with the fluid supplied from the tube 471.

The wire 480 is attached to the second bending portion 412. By attracting the second bending portion 412 from the proximal side A2, the second bending portion 412 is bent.

The first bending portion 411 is bendably driven by the artificial muscle 470. The artificial muscle 470 can more precisely bend the first bending portion 411 in comparison with the wire 480. Accordingly, the artificial muscle 470 is suitable for bendable driving of the first bending portion 411 in which precise operations for treatment are necessary. The artificial muscle 470 is attached to the first bending portion 411 and directly bends the first bending portion 411. Accordingly, it is possible to accurately bend the first bending portion 411 without being affected by the shape on the proximal side of the first bending portion 411.

The second bending portion 412 is bendably driven by the wire 480. The second bending portion 412 does not require a very accurate operation as long as it can greatly bend the shoulder joint 416. Accordingly, it is possible to satisfactorily bend the second bending portion 412 even using the wire 480. Through driving using the wire 480, it is possible to easily secure a longer stroke in comparison with the artificial muscle 470. Accordingly, the wire 480 is suitable for bendable driving of the second bending portion 412 in which a great bending operation is necessary.

With the treatment manipulator 400B according to the present embodiment, it is possible to more efficiently perform observation or treatment. Since the first bending portion 411 which is bendably driven by the artificial muscle 470 can precisely bend the treatment tool, the operator S can more efficiently perform observation or treatment.

While the fourth embodiment of the present invention has been described above in details with reference to the drawings, a specific configuration is not limited to this embodiment and includes a change in design without departing from the gist of the present invention. Elements described in the aforementioned embodiment and modified examples can be appropriately combined into a configuration.

### Modified Example 4-1

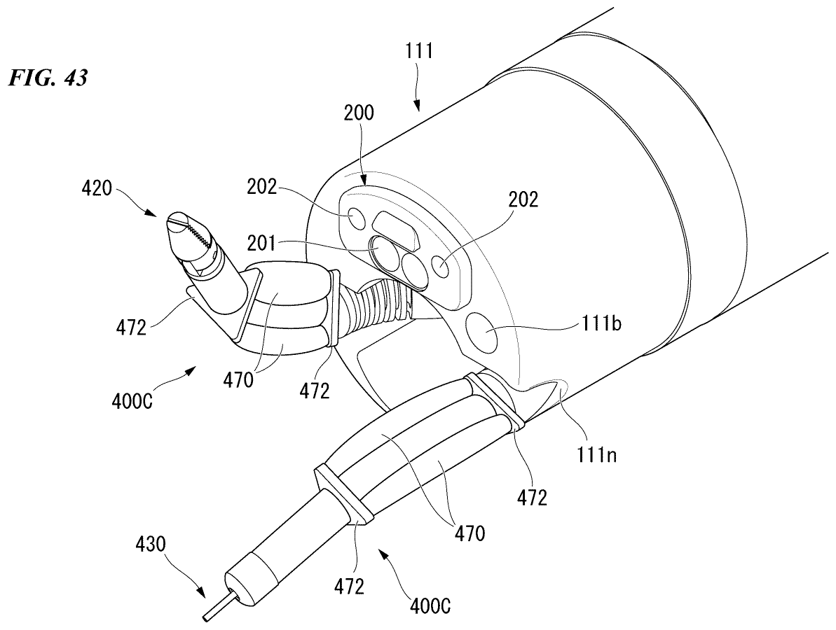

FIG. 43 is a diagram illustrating a treatment manipulator 400C which is a modified example of the treatment manipulator 400B. The treatment manipulator 400C includes a treatment tool and an artificial muscle 470 and does not include a treatment tool arm 410. The artificial muscle 470 is attached to the treatment tool using a ring member 472. A member which is bent with a compression force, for example, a tube or a coil, can be employed instead of the plurality of joint rings 415 illustrated in FIG. 41, and the artificial muscle 470 can be mounted around the member. Instead of a configuration in which the artificial muscle 470 is disposed outside of the joint rings 415, a member which is bent with a compression force, for example, a tube or a coil, may be disposed outside of the artificial muscle 470.

### Modified Example 4-2

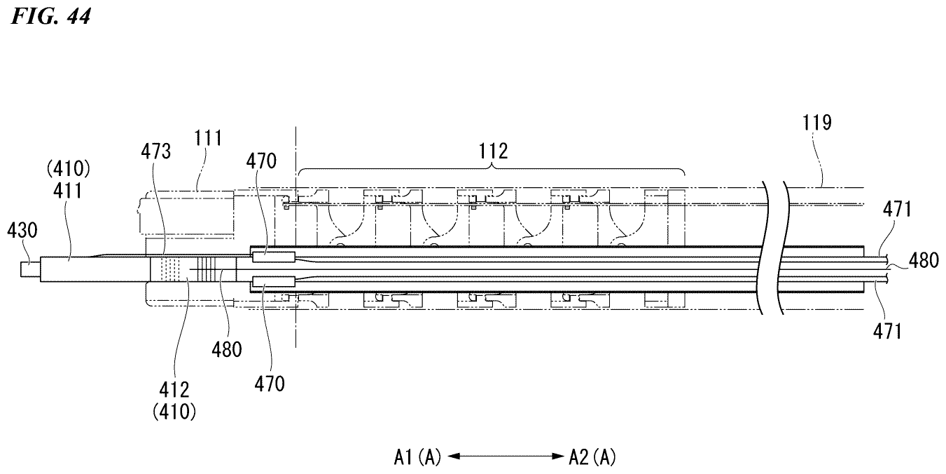

FIG. 44 is a diagram illustrating an artificial muscle 470 which is disposed at a different position.

The artificial muscle 470 may be provided on the proximal side A1 with respect to the second bending portion 412. The first bending portion 411 and the artificial muscle 470 are connected by a distal end wire 473. The artificial muscle 470 bends the first bending portion 411 by causing the distal end wire 473 to move forward and rearward. The artificial muscle 470 illustrated in FIG. 44 is disposed in the distal end portion 111 on the distal end side A1 with respect to the bending portion 112. The artificial muscle 470 is disposed in the distal end portion 111 which is not bent. Accordingly, even when the bending portion 120 is bent, the artificial muscle 470 is not bent.

### Modified Example 4-3

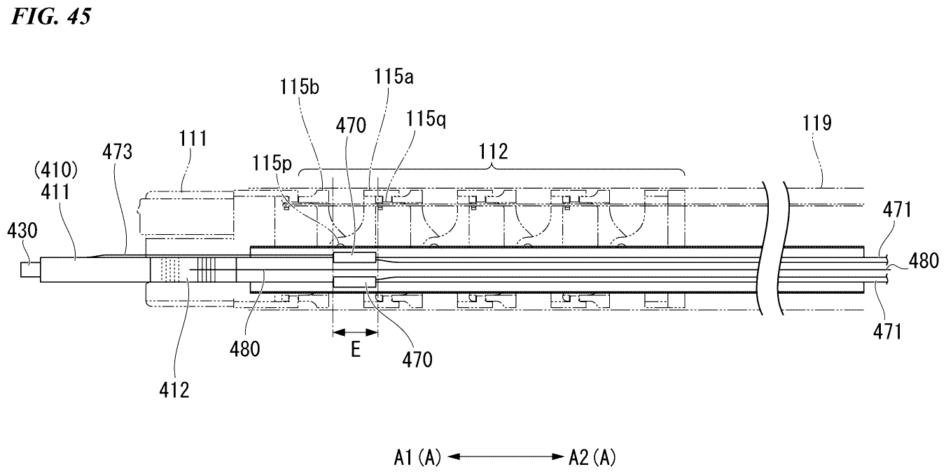

FIG. 45 is a diagram illustrating an artificial muscle 470 which is disposed at a different position.

The artificial muscle 470 illustrated in FIG. 45 is disposed in the bending portion 112. The artificial muscle 470 is disposed in a non-bending area E which is not bent and which is interposed between a first rotary pin 115p and a second rotary pin 115q. Accordingly, even when the bending portion 120 is bent, the artificial muscle 470 is not bent.

### Modified Example 4-4

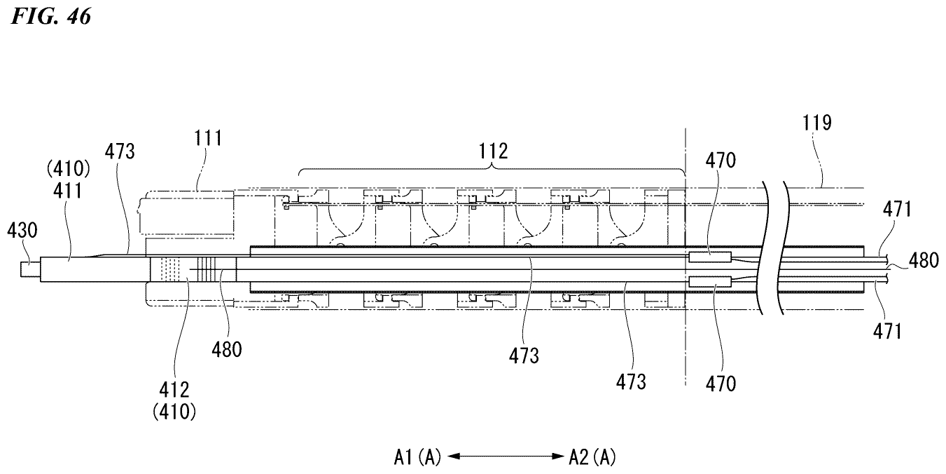

FIG. 46 is a diagram illustrating an artificial muscle 470 which is disposed at a different position.

The artificial muscle 470 illustrated in FIG. 46 is disposed in a flexible portion 119 on the proximal side A2 with respect to the bending portion 112. The artificial muscle 470 is disposed in the flexible portion 119 which is not greatly bent. Accordingly, even when the bending portion 120 is bent, the artificial muscle 470 is not bent.

### Modified Example 4-5

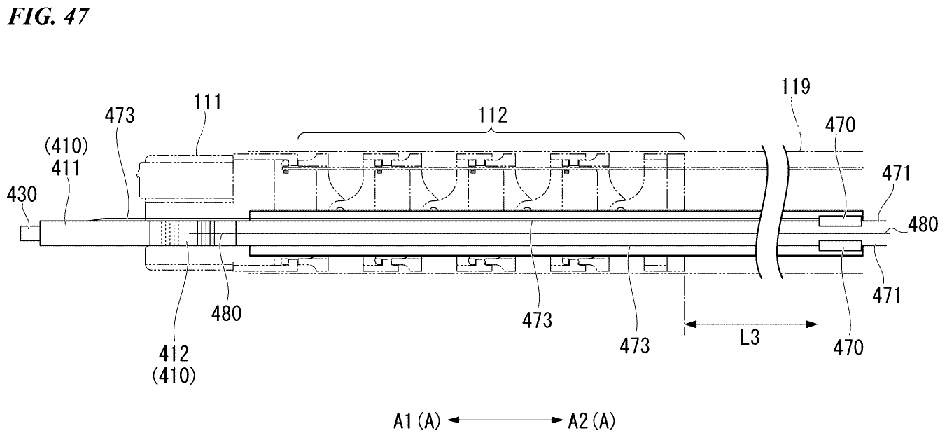

FIG. 47 is a diagram illustrating an artificial muscle 470 which is disposed at a different position.

The artificial muscle 470 illustrated in FIG. 47 is disposed in the flexible portion 119 on the proximal side A2 with respect to the bending portion 112 and is located at a position separated by a distance L3 from the proximal end with respect to the bending portion 112. The distance L3 is longer than a moving distance of the treatment manipulator 400B at the time of treatment. Accordingly, even when the treatment manipulator 400B is intended to move forward and rearward at the time of treatment, the artificial muscle 470 is not inserted into the bending portion 112. Accordingly, even when the bending portion 120 is bent, the artificial muscle 470 is not bent.

### Modified Example 4-6

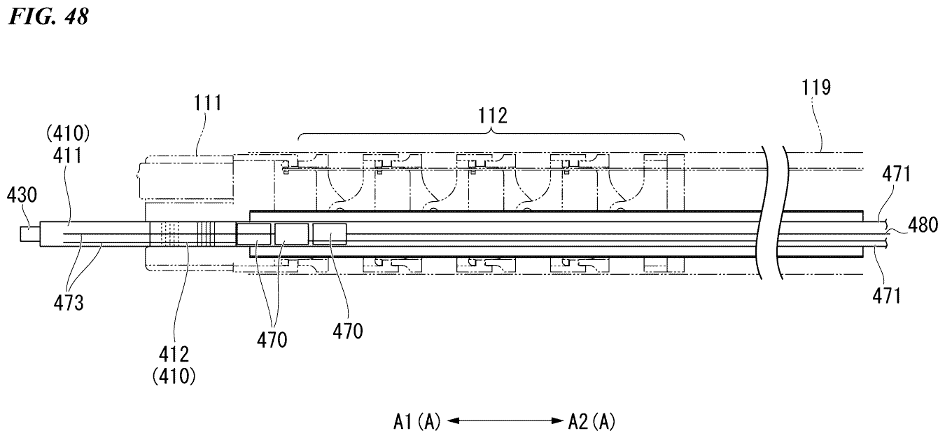

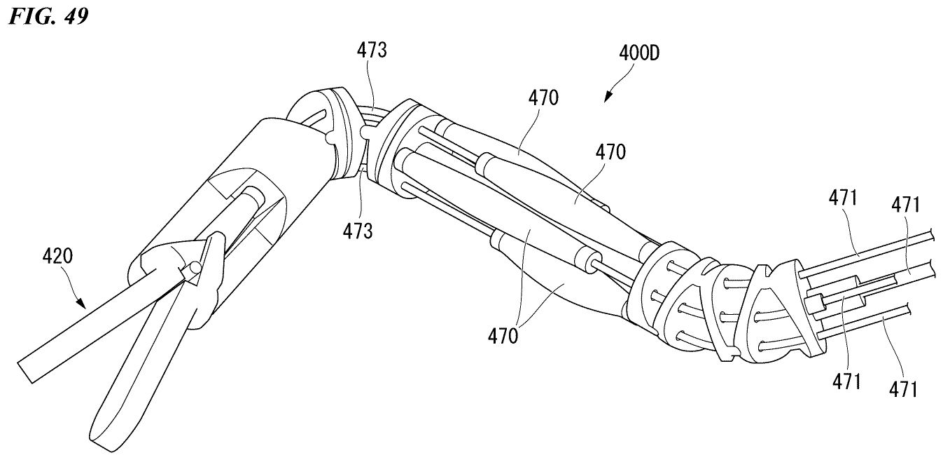

FIG. 48 is a diagram illustrating an artificial muscle 470 which is disposed at a different position.

As illustrated in FIG. 48, a plurality of artificial muscles 470 may be arranged in the axial direction A. It is preferable that some artificial muscles 470 be disposed in a non-bending area. It is not necessary to arrange the plurality of artificial muscles 470 in the radial direction and it is possible to decrease the diameter of the treatment manipulator 400B.

### Modified Example 4-7

FIG. 49 is a diagram illustrating a treatment manipulator 400D which is a modified example of the treatment manipulator 400B. The treatment manipulator 400D does not include a wire 480 and bends all bending portions using the artificial muscle 470. The operator S can precisely bend all the bending portions.

### Fifth Embodiment

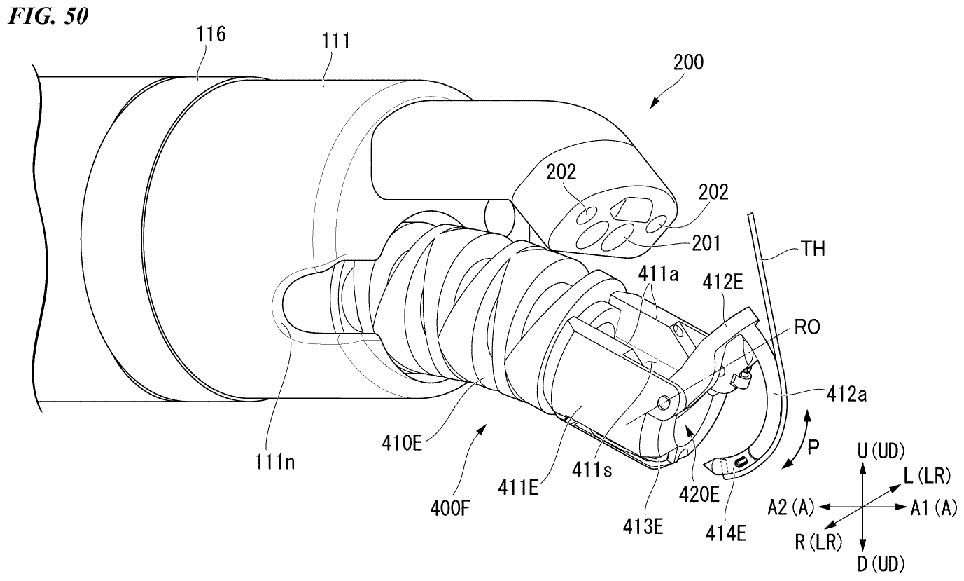

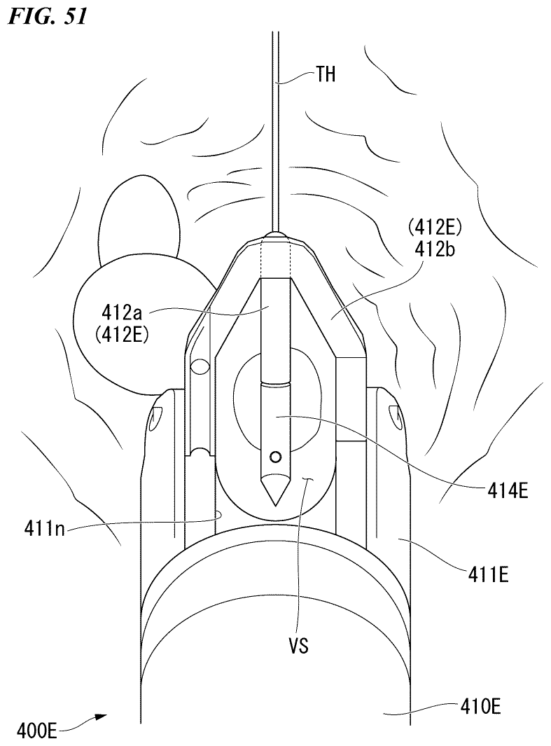

A suture device 400E according to a fifth embodiment of the present disclosure will be described below with reference to FIGS. 50 to 60. In the following description, the same constituents as in the above description will be referred to by the same reference signs, and repeated description thereof will be omitted.

FIG. 50 is a diagram illustrating a suture device 400E that protrudes from the distal end portion 111 of the insertion manipulator 100. The suture device 400E is a device that is inserted into the first channel tube 171 or the like of the insertion manipulator 100 and sutures a defective part D in biological tissue using a thread TH similarly to the treatment manipulator 400 according to the aforementioned embodiment. The suture device 400E has a larger outer diameter than the treatment manipulator 400 and thus is inserted into the first treatment tool lumen 171r instead of the second treatment tool lumen 172r. The suture device 400E is driven by the drive unit (actuator) 550 or the like.

In FIG. 50, the scope 200 is disposed at a position protruding from the distal end portion 111. The scope 200 can also be disposed at a position which is accommodated in the distal end portion 111. That is, the scope 200 can move forward and rearward with respect to the insertion manipulator 100 independently from the suture device 400E. As illustrated in FIG. 50, the scope 200 can be bent toward the center axis of the first channel tube (lumen) 171 in a state in which it has protruded from the distal end portion 111. The scope 200 can be bent toward the viewing field space (the through hole) VS described below.

The suture device 400E includes a hollow bendable arm 410E, a distal end portion 411E that is provided at the distal end of the arm 410E, a first jaw 412E, a second jaw 413E, and a needle 414E.

The arm 410E includes a bending mechanism such as an artificial muscle 470 or a wire 480 similarly to the treatment tool arm 410 of the treatment manipulator 400. The arm 410E includes a hollow portion with a large diameter similarly to the large-diameter treatment tool arm 410A.

The distal end portion 411E is formed in a substantially cylindrical shape. An internal space 411s of the distal end portion 411E communicates with the hollow portion of the arm 410E. The internal space 411s of the distal end portion 411E and the hollow portion of the arm 410E form a channel 420E into which a treatment device such as an anchor applier 400F and a thread attraction tool 400G which will be described later can be inserted. The distal end portion 411E includes a cutout 411n communicating with the internal space 411s on the upper side. A device which is inserted into the channel 420E is not limited thereto, and various treatment tools including a forceps for attracting tissue and applying an appropriate tension thereto, a treatment tool with a screw-shaped (helical) distal end, and a local injection needle for locally injecting a liquid can be inserted thereinto.

The first jaw (an upper jaw portion) 412E and the second jaw (a lower jaw portion) 413E are provided in the distal end portion 411E and can be opened and closed in the vertical direction. In the present embodiment, the first jaw 412E is rotatably attached to the distal end portion 411E, and the second jaw 413E is non-rotatably attached to the distal end portion 411E. A rotary axis RO of the first jaw 412E extends in the lateral direction. The first jaw 412E and the second jaw 413E deliver the needle 414E.

The first jaw 412E includes a jaw body 412b which is formed in a substantially U-shape and a rotary arm 412a which extends in an opening and closing direction P. The needle 414E is detachably attached (latched) to the distal end of the rotary arm 412a.

FIG. 51 is a diagram illustrating a captured image which is captured by the scope 200.

A space surrounded by sides of the jaw body 412b formed in a substantially U-shape communicates with a space surrounded by the cutout 411n of the distal end portion 411E to form a viewing field space (a through hole) VS penetrating the first jaw 412E in the opening and closing direction P. The imaging unit 201 of the scope 200 can image a suturing part or the needle 414E through the viewing field space VS. Even when the first jaw 412E rotates in the opening and closing direction P, the first jaw 412E includes the viewing field space VS and thus does not interfere with the viewing field of the imaging unit 201.

The second jaw (lower jaw portion) 413E is accommodated in the distal end portion 411E. The second jaw 413E may be provided to protrude from the distal end portion 411E to the distal end side A1. The second jaw 413E includes a needle receiving portion 413a and a needle locking mechanism 413b.

The needle receiving portion 413a is a portion that receives the needle 414E attached (latched) to the distal end of the rotary arm 412a of the first jaw 412E.

The needle locking mechanism 413b is a mechanism that locks the needle 414E received in the needle receiving portion 413a. The needle locking mechanism 413b is driven by the drive unit (actuator) 550 or the like.

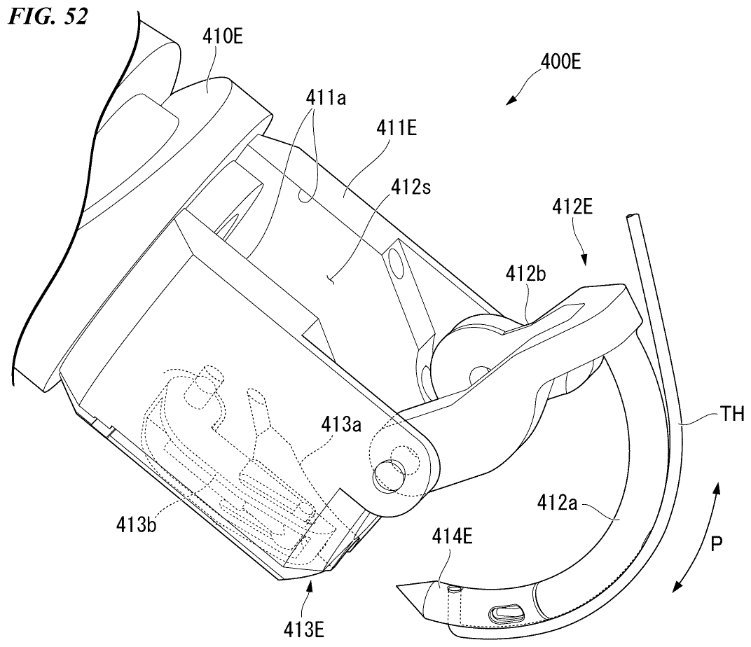

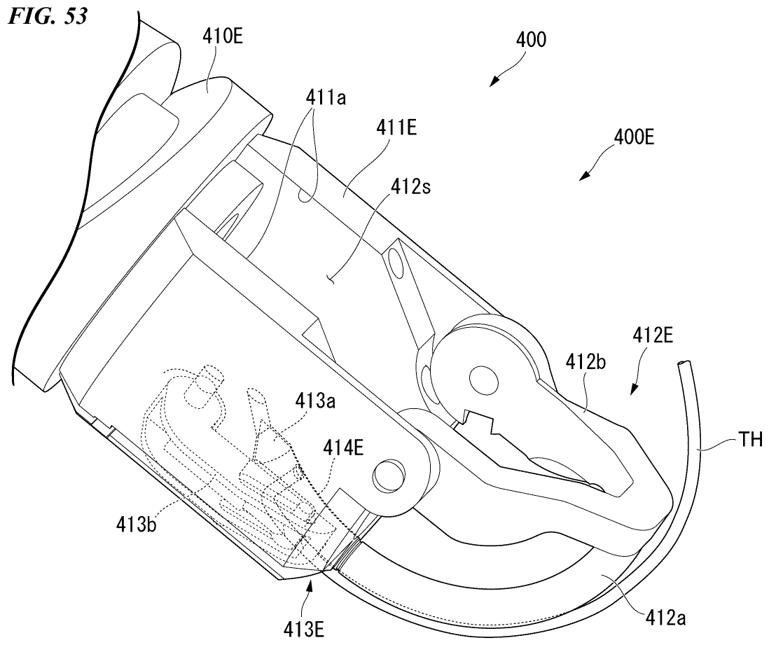

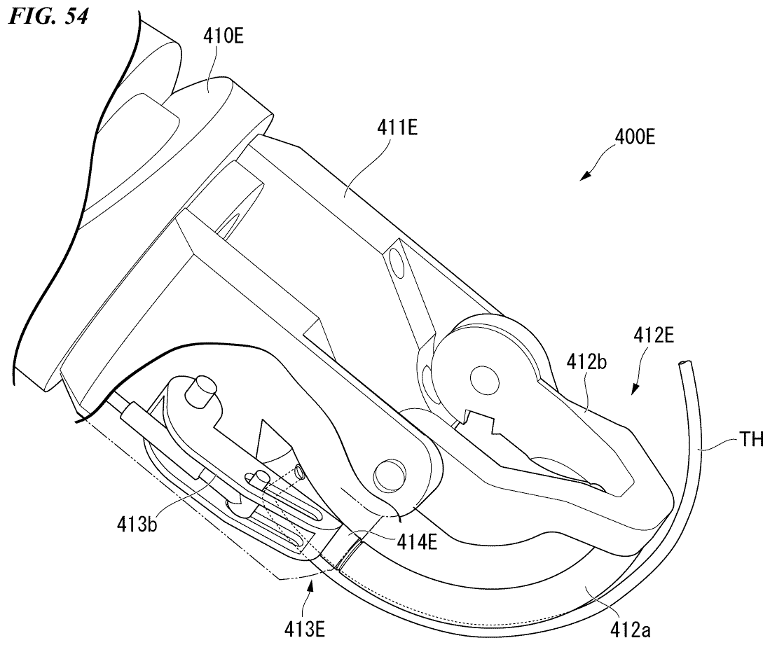

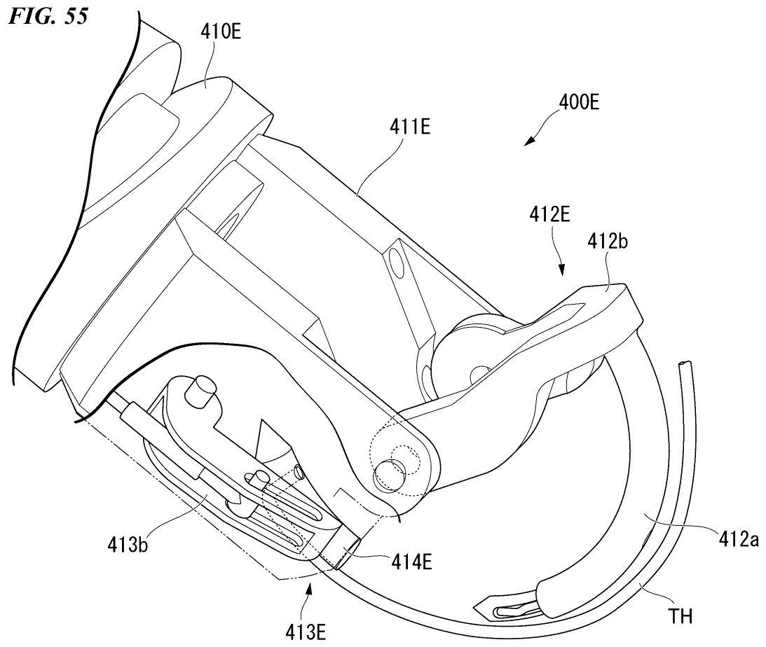

FIGS. 52 to 55 are diagrams illustrating the needle 414E which is delivered from the first jaw 412E to the second jaw 413E. As illustrated in FIG. 52, the rotary arm 412a of the first jaw 412E is closed downward. As illustrated in FIG. 53, the needle 414E is received in the needle receiving portion 413a. As illustrated in FIG. 54, the needle locking mechanism 413b locks the needle 414E. At this time, the needle locking mechanism 413b releases connection between the rotary arm 412a and the needle 414E. As illustrated in FIG. 55, the rotary arm 412a of the first jaw 412E is opened upward. The needle 414E departs from the rotary arm 412a of the first jaw 412E and is located in the needle receiving portion 413a.

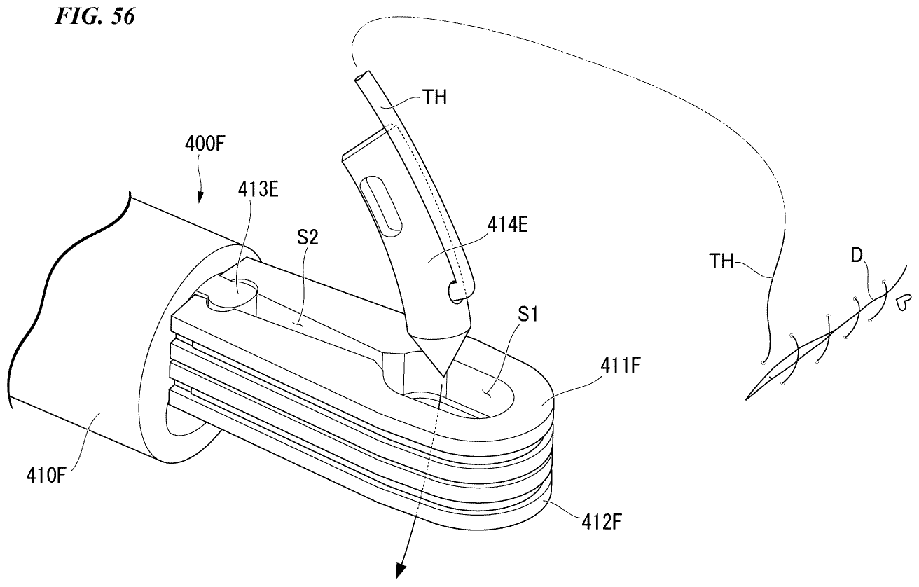

FIG. 56 is a diagram illustrating a sutured defective part D.

The operator S pierces a circumferential edge of the defective part D with the rotary arm 412a to which the needle 414E is attached (latched) and puts the thread TH on the circumferential edge of the defective part D. As illustrated in FIG. 56, the operator S inserts the anchor applier 400F into the channel 420E after putting the thread TH on the circumferential edge of the defective part D.

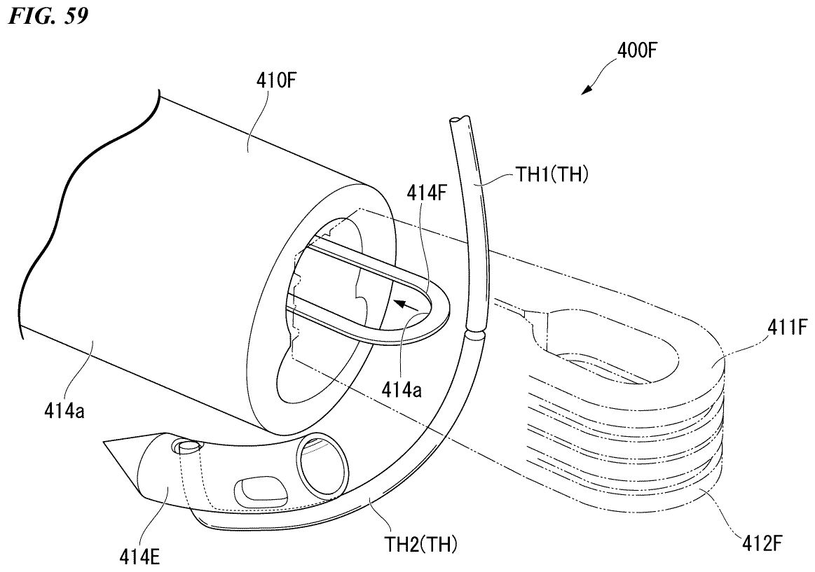

The anchor applier 400F can be inserted into the channel 420E of the suture device 400E and protrude from the channel 420E to the distal end side A1. The anchor applier 400F includes a long body 410F, a first anchor 411F, a second anchor 412F, a plug 413F, and a blade 414F (see FIG. 59).

The first anchor 411F and the second anchor 412F are detachably attached to the distal end of the body 410F. The first anchor 411F and the second anchor 412F are provided in parallel in the vertical direction. The first anchor 411F and the second anchor 412F have the same shape. The first anchor 411F (and the second anchor 412F) is formed in a substantially U-shape, and a distal end space (a needle hole) S1 into which the needle 414E can be inserted and a proximal space (a thread hole) S2 into which a thread TH can be inserted are formed therein. The distal end space S1 and the proximal space S2 communicate with each other.

The plug 413F is detachably provided in the proximal space S2. When the plug 413F is disposed in the proximal space S2, the first anchor 411F and the second anchor 412F are pressed outward by the plug 413F, and the proximal space S2 is widened. At this time, the first anchor 411F and the second anchor 412F are not fixed to the thread TH.

The blade 414F is provided to move forward and rearward on the distal end side A1 of the body 410F. The blade 414F is provided to move forward and rearward between the first anchor 411F and the second anchor 412F. The blade 414F includes an edge 414a (see FIG. 59) and can cut the thread TH.

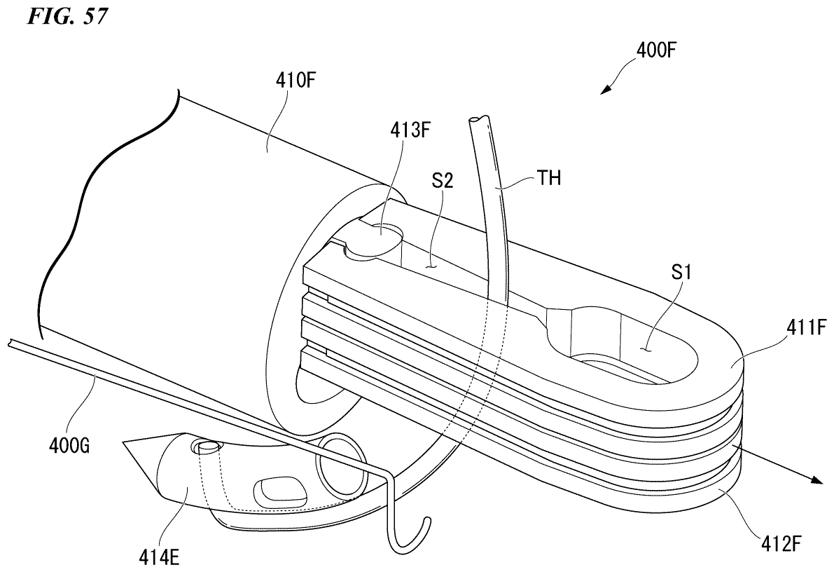

FIGS. 57 to 60 are diagrams illustrating operations of the anchor applier 400F.

As illustrated in FIG. 56, the operator S causes the needle 414E to pass through the distal end space S1 of the anchor applier 400F protruding from the channel 420E to the distal end side A1. The thread TH is inserted into the distal end space S1.

As illustrated in FIG. 57, the operator S inserts the thread attraction tool 400G into the channel 420E. The anchor applier 400F and the thread attraction tool 400G can be simultaneously inserted into the channel 420E. For example, the thread attraction tool 400G includes a hooking portion with a hook shape or a helical coil shape at its distal end. The operator S attracts the thread TH to the proximal side A2 using the thread attraction tool 400G and moves the thread TH from the distal end space S1 to the proximal space S2.

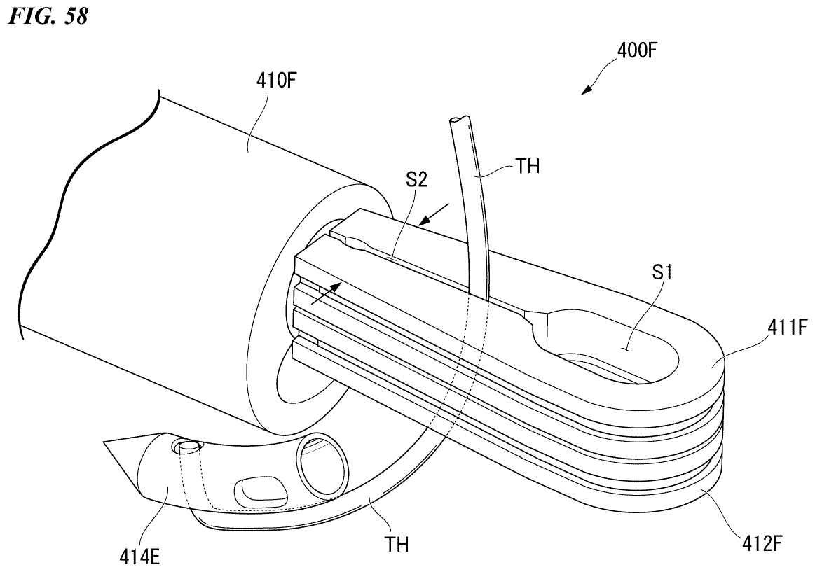

As illustrated in FIG. 58, the operator S attracts the plug 413F to the proximal side A2 and pulls the plug 413F from the first anchor 411F and the second anchor 412F. The proximal space S2 of the first anchor 411F is narrowed and puts the thread TH therebetween. The first anchor 411F is fixed to a first fixed portion of the thread TH which is inserted into the proximal space S2. The proximal space S2 of the second anchor 412F is narrowed and puts the thread TH therebetween. The second anchor 412F is fixed to a second fixed portion of the thread TH which is inserted into the proximal space S2.

As illustrated in FIG. 59, the operator S attracts the blade 414F to the proximal side A2 and cuts the thread TH. The blade 414F cuts the thread TH between the first fixed portion and the second fixed portion. The thread TH on the defective part D side which is one end of the cut thread TH is referred to as a first thread TH1. The thread TH on the needle 414E side which is the other end of the cut thread TH is referred to as a second thread TH2. The first anchor 411F is attached to the first thread TH1. The second anchor 412F is attached to the second thread TH2. The operator S removes the anchor applier 400F and the thread attraction tool 400G from the channel 420E.

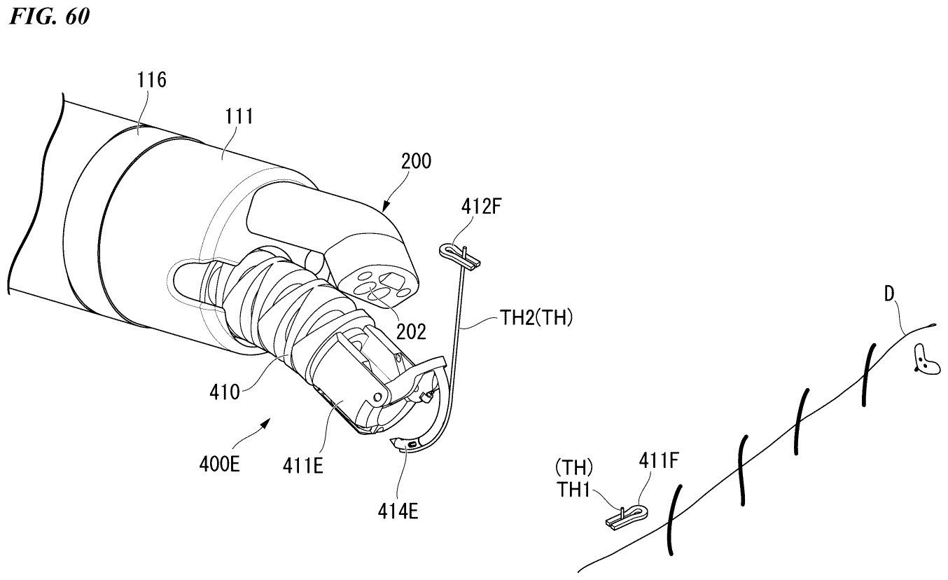

As illustrated in FIG. 60, the first anchor 411F attached to the first thread TH1 serves as a thread stopper of ligation (termination) of the first thread TH1 suturing the defective part D. The second anchor 412F attached to the second thread TH2 serves as a thread stopper of a start end of next suturing. The operator S can adjust a length of the second thread TH2 which is used for next suturing by adjusting an amount of attraction of the thread TH using the thread attraction tool 400G and adjusting a length the thread TH between the first fixed portion and the second fixed portion.

With the suture device 400E according to the present embodiment, it is possible to more efficiently perform observation or treatment. The operator S can successively perform installation of the first anchor 411F serving as an end of suturing, attraction of the thread TH, and installation of the second anchor 412F serving as a start of next suturing in a lumen. Accordingly, it is not necessary to perform preparation of thread stoppage or a complex operation using a knot pusher and it is possible to easily perform suturing to shorten a suturing time.

While the fifth embodiment of the present invention has been described above in details with reference to the drawings, a specific configuration is not limited to this embodiment and includes a change in design without departing from the gist of the present invention. Elements described in the aforementioned embodiment and modified examples can be appropriately combined into a configuration.

Some or all of the aforementioned functions may be realized by recording a program in each embodiment on a computer-readable recording medium and causing a computer system to read and execute the program recorded on the recording medium. The “computer system” includes an operating system (OS) or hardware such as peripherals. The “computer-readable recording medium” is a portable medium such as a flexible disk, a magneto-optical disc, a ROM, or a CD-ROM or a storage device such as a hard disk incorporated into a computer system. The “computer-readable recording medium” may include a medium that dynamically holds a program for a short time such as a communication line when the program is transmitted via a network such as the Internet or a communication circuit line such as a telephone line and a medium that holds a program for a predetermined time such as a volatile memory in a computer system serving as a server or a client in that case. The program may be a program for realizing some of the aforementioned functions. The program may be a program for realizing the aforementioned functions in combination with another program stored in advance in the computer system.

### Appendix 1

An anchor applier including:
a main body;
a first anchor and a second anchor detachably attached to the main body;
a plug detachably attached to the first anchor and the second anchor; and
a blade,
wherein a thread hole configured to insert a thread is formed in the first anchor and the second anchor,
the first anchor is fixed to a first fixing part of the thread that passes through the thread hole when the plug is removed,
the second anchor is fixed to a second fixing part of the thread that passes through the thread hole when the plug is removed, and
the blade is capable of cutting the thread between the first fixing part and the second fixing part.

### Appendix 2

The anchor applier according to appendix 1, wherein the first anchor and the second anchor are attached side by side to a distal end of the main body.

### Appendix 3

The anchor applier according to appendix 1, wherein a needle hole is formed in the first anchor and the second anchor, configured to insert a needle to which the thread is attached, and the needle hole is connected to the thread hole.

### Appendix 4

The anchor applier according to appendix 1 wherein
the plug is attached to the first anchor to expand the thread hole of the first anchor, and
the plug is attached to the second anchor to expand the thread hole of the second anchor.

### Appendix 5

The anchor applier according to appendix 1, wherein the blade is provided on a distal end side of the main body so as to be cable of advancing and retracting.

### Appendix 6

The anchor applier according to appendix 5, wherein the blade is provided so as to be capable of advancing and retracting between the first anchor and the second anchor.

### Appendix 7

A method for placing a first anchor and a second anchor in which a thread hole configured to insert a thread is formed, the method including:
passing the thread through the thread hole of the first anchor and the thread hole of the second anchor;
fixing the first anchor to a first fixing part of the thread that passes through the thread hole;
fixing the second anchor to a second fixing part of the thread that passes through the thread hole; and
placing the first anchor by cutting the thread between the first fixing part and the second fixing part.

### Appendix 8

The anchor placement method according to appendix 7, wherein
the first anchor and the second anchor have a needle hole formed therein through which a needle to which the thread is attached can be inserted and which communicates with the thread hole,
the needle to which the thread is attached is passed through the needle hole, and
the thread is moved from the needle hole to the thread hole.

### Appendix 9

The anchor placement method according to appendix 7, wherein
the first anchor is fixed to the first fixing part by narrowing the thread hole of the first anchor, and
the second anchor is fixed to the second fixing part by narrowing the thread hole of the second anchor.

### Appendix 10

The anchor placement method according to appendix 7, wherein a length of the thread between the first fixing part and the second fixing part is adjusted by retracting the thread before cutting the thread.

The present invention can be applied to a medical system that performs observation and treatment in a hollow organ.

What is claimed is:

1. A medical system comprising a suture device,
wherein the suture device includes:
an arm;
a first jaw and a second jaw that are attached to a distal end of the arm; and
a needle configured to attach a thread, and
wherein at least one of the first jaw and the second jaw configured to be opened and closed to deliver the needle therebetween.

2. The medical system according to claim 1, further comprising a medical manipulator including a lumen which configured to insert the suture device and a scope including an imaging unit.

3. The medical system according to claim 2, wherein the first jaw configured to be opened and closed and includes a through hole going through in the opening and closing direction, and
wherein the scope configured to image the needle through the through hole.

4. The medical system according to claim 3, wherein the scope is bendable in a direction directed to the through hole in a state in which the scope has protruded from a distal end portion of the medical manipulator.

5. The medical system according to claim 3, wherein the arm includes a cutout at a distal end of the arm, and
wherein the cutout communicates with the through hole and forms a part of the through hole.

6. The medical system according to claim 1, wherein the suture device includes a channel configured to insert a treatment device, and
wherein the treatment device configured to protrude from a distal end of the channel to the distal end side.

7. The medical system according to claim 1, wherein the arm includes a bendable bending portion.

8. The medical system according to claim 2, wherein the medical manipulator includes a bendable bending portion.

9. The medical system according to claim 6, further comprising an anchor applier as the treatment device,
wherein the anchor applier includes:
a body;
a first anchor and a second anchor that are detachably attached to the body;
a plug that is detachably attached to the first anchor and the second anchor; and
a blade,
wherein a thread hole configured to insert a thread is formed in the first anchor and the second anchor,
wherein the first anchor is fixed to a first fixed portion of the thread inserted into the thread hole when the plug is detached,
wherein the second anchor is fixed to a second fixed portion of the thread inserted into the thread hole when the plug is detached, and
wherein the blade configured to cut the thread between the first fixed portion and the second fixed portion.

10. The medical system according to claim 9, wherein the first anchor and the second anchor are attached to a distal end of the body in parallel.

11. The medical system according to claim 9, wherein a needle hole configured to insert a needle with the thread attached thereto is formed in the first anchor and the second anchor, and
wherein the needle hole communicates with the thread hole.

12. The medical system according to claim 9, wherein the plug is attached to the first anchor to widen the thread hole of the first anchor, and
wherein the plug is attached to the second anchor to widen the thread hole of the second anchor.

13. The medical system according to claim 9, wherein the blade is provided to be movable forward and rearward on the distal end side of the body.

14. The medical system according to claim 13, wherein the blade is provided to be movable forward and rearward between the first anchor and the second anchor.

15. The medical system according to claim 6, further comprising a thread attraction tool as the treatment device,
wherein the thread attraction tool includes a hooking portion with a hook shape or a helical coil shape at a distal end.

16. A suture device comprising:
an arm;
a first jaw and a second jaw that are attached to a distal end of the arm; and
a needle configured to attach a thread,
wherein at least one of the first jaw and the second jaw configured to be opened and closed to deliver the needle therebetween.

17. The suture device according to claim 16, wherein the first jaw configured to be opened and closed and includes a through hole going through in the opening and closing direction.

18. The suture device according to claim 17, wherein the arm includes a cutout at a distal end of the arm, and
wherein the cutout communicates with the through hole and forms a part of the through hole.

19. The suture device according to claim 16, wherein the suture device includes a channel configured to insert a treatment device, and
wherein the treatment device configured to protrude from a distal end of the channel to the distal end side.

20. The suture device according to claim 16, wherein the arm includes a bendable bending portion.
